(19) United States
(12) Patent Application Publication
(10) Pub. No.: US 2008/0194416 A1
(43) Pub. Date: Aug. 14, 2008

(54) DETECTION OF MATURE SMALL RNA MOLECULES

(75) Inventor: Fuqiang Chen, St. Louis, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: SIGMA ALDRICH, St. Louis, MO (US)

(21) Appl. No.: 11/672,591

(22) Filed: Feb. 8, 2007

(51) Int. Cl. C40B 30/04; C12Q 1/68

(52) U.S. Cl. 506/9; 435/6

(57) ABSTRACT

Methods, compositions, and kits for detecting mature small RNAs are provided herein. The methods comprise ligating at least one linker to a mature small RNA in the presence of a complementary ligation template to generate a ligation product. The ligation product is a hybrid molecule comprising the linker and the mature small RNA.

### FIELD OF THE INVENTION

The present invention provides methods, compositions, and kits for detecting mature small RNA molecules.

### BACKGROUND OF THE INVENTION

Small RNAs, such as microRNAs (miRNAs) or short interfering RNAs (siRNAs), regulate gene expression by targeting messenger RNAs for cleavage or translational repression, or altering transcription by silencing genes, interfering with RNA splicing or processing, or affecting chromatin structure. Small RNAs, therefore, play critical roles in cell proliferation, cell differentiation, and cellular responses, and their misexpression or misregulation is involved in some disease states (Jin et al. (2004) Nature Neurosci. 7: 113-117; Michael et al. (2003) Mol Cancer Res. 1:882-891). Small RNAs are generated by specific enzyme complexes from much larger RNA precursors, and a mature small RNA has several key characteristic features such as a small size (generally about 20-30 nucleotides), a 5′ terminal monophosphate, and a 3′ terminal hydroxyl group. Attempts to detect, quantify, and analyze mature small RNAs have been hindered by their small sizes and, sometimes, attendant low copy numbers.

Northern blotting has been used to detect mature small RNAs, but this method suffers from poor sensitivity. Likewise, nucleic acid microarrays have been used to quantify mature small RNAs, but this method also requires a high concentration of input target for efficient hybridization. The small size of mature small RNAs precludes their amplification by quantitative or reverse transcriptase PCR (although the larger precursors may be PCR amplified). Methods have been developed to facilitate PCR detection of mature small RNAs. For example, mature small RNAs have been lengthened by the addition of at least one oligonucleotide adaptor. Alternatively, probes comprising a portion that hybridizes to a small RNA are ligated together and then used for PCR amplification. These methods are less than ideal because the ligation of single stranded molecules is inefficient and/or the small RNA is not directly detected. Thus, a need still exists for a sensitive, quick, cost-effective method for the direct detection of mature small RNAs.

### SUMMARY OF THE INVENTION

One aspect of the present invention provides a method for detecting a mature small RNA whose sequence is known. The method comprises providing a sample that includes the mature small RNA. A 5′ linker is ligated to the 5′ end of the mature small RNA in the presence of a complementary ligation template that spans the ligation junction, whereby a ligation product is formed. The ligation product is a hybrid molecule comprising the 5′ linker and the mature small RNA. The ligation product can then be assayed, such that the mature small RNA is detected.

Another aspect of the invention provides a method for detecting a population of mature small RNAs whose sequences are known or unknown. The method comprises providing a sample that includes a population of mature small RNAs. A first linker is ligated to one end of a mature small RNA in the population of mature small RNAs in the presence of a first semi-degenerate ligation template that spans the first ligation junction, whereby a plurality of first ligation products is formed. The first ligation products are hybrid molecules comprising the first linker and a mature small RNA. A second linker is ligated to the small RNA end of a ligation product in the plurality of first ligation products in the presence of a second semi-degenerate ligation template that spans the second ligation junction, whereby a plurality of second ligation products is formed. The second ligation products are hybrid molecules comprising the first linker, a mature small RNA, and the second linker. The second ligation products can then be assayed, such that a population of mature small RNAs is detected.

A further aspect of the invention is a kit for detecting a mature small RNA whose sequence is known. The kit comprises a 5′ linker for ligating to the 5′ end of the mature small RNA, a ligation template that is complementary to the junction between the 5′ linker and the mature small RNA, a ligase, and instructions for using the kit.

Yet another aspect of the invention is a kit for detecting a population of mature small RNAs whose sequences are known or unknown. The kit comprises a 5′ linker for ligating to the 5′ end of a mature small RNA, a 3′ linker for ligating to the 3′ end of a mature small RNA, a 5′ semi-degenerate ligation template that is complementary to the 5′ ligation junction, a 3′ semi-degenerate ligation template that is complementary to the 3′ ligation junction, a ligase, and instructions for using the kit.

Other aspects and features of the invention are described in more detail below.

### DESCRIPTION OF THE FIGURES

FIG. 1 is schematic diagram illustrating the method for detecting a mature small RNA. The mature small RNA is represented by the white bar, the 5′ linker by the striped bar, and the complementary ligation template by the stippled bar.

FIG. 2 is schematic diagram illustrating the method for detecting a population of mature small RNAs using one ligation step. The population of mature small RNAs is represented by the gray bar. The 5′ linker is represented by the black bar, and the 3′ linker is represented by the white bar. The constant regions of the 5′ and 3′ degenerate ligation templates that hybridize with the 5′ and 3′ linkers are represented by black and white bars, respectively. The degenerate regions of the 5′ and 3′ degenerate ligation templates that hybridize with the small RNAs are represented by the repeat of “Ns” (N corresponds to any nucleotide).

FIG. 3 is schematic diagram illustrating the method for detecting a population of mature small RNAs using two ligation steps. The population of mature small RNAs is represented by the gray bar. The 5′ linker is represented by the black bar, and the 3′ linker is represented by the white bar. The constant regions of the 5′ and 3′ degenerate ligation templates that hybridize with the 5′ and 3′ linkers are represented by black and white bars, respectively. The degenerate regions of the 5′ and 3′ degenerate ligation templates that hybridize with the small RNAs are represented by the repeat of “Ns” (N corresponds to any nucleotide).

### DETAILED DESCRIPTION OF THE INVENTION

The present invention provides methods, compositions, and kits for the detection of mature small RNAs. The methods of the invention take advantage of the 5′ terminal phosphate of a mature small RNA. Ligation of a 5′ linker to the 5′ end of a mature small RNA is catalyzed by a template-dependent ligase in the presence of a complementary ligation template. A ligation reaction catalyzed by a template-dependent ligase is much faster and more efficient than one involving single-stranded nucleic acids. The ligation product comprising the linker and the mature small RNA can then be directly assayed to detect and quantify the mature small RNA. It has also been discovered that the methods of the invention may be modified to include two linkers, and two semi-degenerate ligation templates, whereby previously unknown mature small RNAs may be detected and identified.

### (I) Method for Detecting a Mature Small RNA

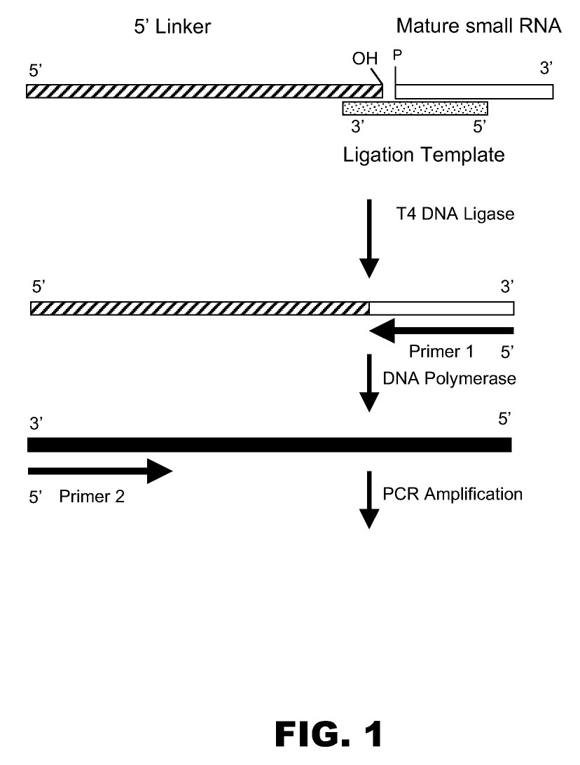

One aspect of the present invention provides a method for detecting a mature small RNA whose nucleotide sequence is known. The method comprises providing a sample that includes the mature small RNA to be detected. The method further comprises ligating a 5′ linker to the 5′ end of the mature small RNA in the presence of a complementary ligation template (see FIG. 1). The ligation template spans the ligation junction, such that the hydroxyl group at the 3′ end of the linker lies in close proximity to the phosphate group at the 5′ end of the mature small RNA. A ligase catalyzes the formation of a phosphodiester bond between the two adjacent nucleotides, such that the 5′ linker and the mature small RNA are joined and a ligation product is formed. The method further comprises assaying the ligation product such that the mature small RNA is detected. Example 1 demonstrated that this ligation-based method could readily detect a phosphorylated small RNA, but not an unphosphorylated small RNA.

(a) Providing a Sample Comprising a Mature Small RNA

The source of a small RNA-containing sample that is suitable for use in this invention can and will vary depending upon the application. The sample comprising a mature small RNA may be derived from animals, plants, fungi, protists, viruses, bacteria, or archaea. Likewise, the sample derived from any of the aforementioned sources may range from a preparation of essentially pure RNA molecules to a crude extract of a cell. In one embodiment, the sample may be an isolated preparation of small RNA molecules. In another embodiment, the sample may be an isolated preparation of total RNA extracted from a cell. In yet another embodiment, the sample may be a cytosolic cellular extract comprising nucleic acids, proteins, lipids, and carbohydrates. In still another embodiment, the sample may be an intact cell. In yet another embodiment, the sample comprising the small RNA may be an in vitro transcription reaction or a chemical synthesis reaction. Total RNA or small RNA may be isolated and purified from cells, cellular extracts, or in vitro reactions using commercially available kits or techniques well known in the art (for reference, see Ausubel et al. (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y.).

Several different types of small RNA molecules may be detected by the method of the invention. Examples of mature small RNAs that may be detected include, but are not limited to, microRNA (miRNA), short interfering RNA (siRNA), repeat-associated siRNA (rasiRNA), transacting siRNA (tasiRNA), Piwi-interacting RNA (piRNA), and 21U-RNA. The small RNA may be encoded in the genome. For example, miRNAs are derived from longer noncoding transcripts and rasiRNAs are generally derived from repetitive genomic sequences. Alternatively, the small RNA may originate from an exogenous double-stranded RNA molecule. For example, many siRNAs are derived from exogenous RNAs. Regardless of its origination, each of the different types of small RNA is generated from a larger precursor by a specific endonuclease enzyme complex. The larger precursor may be double-stranded, or it may be a single-stranded transcript that forms a hairpin or stem-loop structure. Regardless of the structure of the larger precursor, each is generally cleaved into at least one mature small RNA product by the endonuclease complex. As a consequence, mature small RNA molecules have several characteristic features including a 5′ terminal phosphate and a 3′ terminal hydroxyl group.

The length of the mature small RNA that may be detected by the method can and will vary. In one embodiment, the mature small RNA may range from about 10 nucleotides to about 50 nucleotides in length. In another embodiment, the mature small RNA may range from about 15 nucleotides to about 35 nucleotides in length. In preferred embodiments, the mature small RNA may range from about 17, 18, 19, 20, 21, 22, 23, 24, 25, 26, 27, 28, 29, 30, 31, or 32 nucleotides in length.

The amount of small RNA in the sample added to a ligation reaction can and will vary depending upon the source of the RNA-containing sample. In general, any amount of RNA that can be ligated to a linker may be utilized. Typically, the amount of highly purified small RNA used per reaction volume will be less than the amount of total RNA used per reaction volume. As demonstrated in the examples, about 50 nanogram (ng) to about 300 ng of purified small RNA (per 10 μl reaction mixture) was optimal, while about 100 ng to about 400 ng of total RNA (per 10 μl reaction mixture) was optimal.

(b) Ligating a 5′ Linker to the Mature Small RNA

The method of the invention further comprises ligating a 5′ linker to the 5′ end of the mature small RNA in the presence of a complementary ligation template. The complementary ligation template facilitates ligation by juxtaposing the 3′ end of the linker and the 5′ end of the small RNA.

(i) 5′ Linker

During the method of the invention, a nucleic acid (the 5′ linker) is attached to the 5′ end of a mature small RNA. The 5′ linker has a free hydroxyl group at its 3′ end. The constituents of the linker can and will vary depending upon the application. In one embodiment, the 5′ linker may be a DNA polynucleotide. In an alternate embodiment, the 5′ linker may be a chimeric DNA-RNA nucleic acid in which a DNA polynucleotide is modified to have at least one ribonucleotide at the 3′ end.

The nucleotides of the 5′ linker may be standard (i.e., adenosine, guanosine, cytidine, thymidine, and uridine), as well as nonstandard nucleotides. Non-limiting examples of nonstandard nucleotides include inosine, xanthosine, isoguanosine, isocytidine, diaminopyrimidine, and deoxyuridine. The 5′ linker may comprise modified or derivatized nucleotides. Non-limiting examples of modifications on the ribose or base moieties include the addition (or removal) of acetyl groups, amino groups, carboxyl groups, carboxymethyl groups, hydroxyl groups, methyl groups, phosphoryl groups, and thiol groups. In particular, included are 2′-O-methyl and locked nucleic acid (LNA) nucleotides. Suitable examples of derivatized nucleotides include those with covalently attached dyes, such as fluorescent dyes or quenching dyes, or other molecules, such as biotin, digoxygenin, or a magnetic particle. The 5′ linker may comprise synthetic nucleotide analogs, such as morpholinos or peptide nucleic acids (PNA). Phosphodiester or phosphothioate bonds may link the nucleotides or nucleotide analogs of the 5′ linker.

The length of the 5′ linker can and will vary depending upon, for example, the desired length of the ligation product, costs of synthesizing the linker, and desired features of the 5′ linker. In general, the 5′ linker will be at least about 15 nucleotides in length, but it may be up to several hundred nucleotides in length. As shown in the examples, the 5′ linker may range from about 90 nucleotides to about 100 nucleotides in length.

The 5′ linker may also assume a secondary structure. The secondary structure may comprise helices, hairpins, base-paired regions, base-paired stems, unpaired loops, unpaired bulges, and single-stranded regions. The base pairing may be standard Watson-Crick base pairing, or the base pairing may be non-standard base paring (e.g., between G and U). In one embodiment, the 5′ linker may comprise a stem-loop structure, which presumably imparts increased stability to the 5′ linker. In such a linker, the stem comprises the 5′ end of the linker and a region near the 3′ end of the linker, with the 3′ end being single-stranded. The stem may range from about 3 base pairs to about 20 base pairs in length, and more preferably from about 8 base pairs to about 10 base pairs. The loop may range from about 30 nucleotides to about 200 nucleotides, and more preferably from about 65 nucleotides to about 75 nucleotides. The single-stranded 3′ region may range from about 2 nucleotides to about 30 nucleotides in length, and more preferably from about 6 nucleotides to about 12 nucleotides in length.

Additional sequences may be included in the 5′ linker for use in downstream applications. For example, the 5′ linker may comprise at least one restriction endonuclease recognition site. Alternatively, the 5′ linker may further comprise at least one RNA polymerase promoter site. Non-limiting examples of promoter sites include those for T3, T7, or SP6 RNA polymerases.

The 5′ linker may be free in solution, such that the small RNA is detected in solution. Alternatively, the 5′ linker may be attached to a solid support, whereby the 3′ terminal end is free. Thus, in the latter embodiment, the mature small RNA is attached to the linker that is attached to the solid support. Non-limiting examples of a suitable solid support include a glass surface, a silica surface, a plastic surface, a polymer surface, a co-polymer surface, or a metal surface.

In a preferred embodiment, the 5′ linker is about 90-100 nucleotides in length and comprises deoxyribonucleotides with a single ribonucleotide at the 3′ terminal end. The 5′ linker forms a stem-loop structure; the stem is about 8-10 bp in length and comprises the 5′ end of the linker and a complementary region near the 3′ end of the linker, with an intervening loop of about 68-70 nucleotides. The terminal 6-10 nucleotides at the 3′ end of the linker are single-stranded (and are complementary to part of the ligation template, see below). The 5′ linker has a 3′ terminal hydroxyl group for ligation to the 5′ terminal phosphate group of a mature small RNA.

(ii) Complementary Ligation Template

Ligation of the 5′ linker to the 5′ end of the mature small RNA is performed in the presence of a complementary ligation template. The ligation template comprises two distinct regions: a 5′ region that hybridizes under stringent condition with the 5′ end of the mature small RNA and a 3′ region that hybridizes under stringent condition with the 3′ end of the 5′ linker. To hybridize under stringent conditions, the complementary ligation template may be an exact complement or it may be a nearly exact complement of its two (known) target sequences. Since the ligation template hybridizes to both the 5′ linker and the mature small RNA, it spans the ligation junction, such that the 3′ end of linker is brought into close proximity to the 5′ end of the mature small RNA.

In general, the complementary ligation template will be at least about 10 nucleotides in length, with about half of the ligation template having complementarity to the mature small RNA and the other half having complementarity to the 5′ linker. A person skilled in the art will appreciate that the complementary ligation template may be longer, provided it hybridizes with both the small RNA and the 5′ linker. As demonstrated in the examples, the complementary ligation template may range from about 14 nucleotides to about 24 nucleotides in length, with about 7 nucleotides to about 12 nucleotides at the 5′ end that hybridize with the mature small RNA, and about 7 nucleotides to about 12 nucleotides at the 3′ end that hybridize with the 5′ linker.

The constituents of the ligation template can and will vary depending upon the application. In one embodiment, the ligation template may be an RNA oligonucleotide. In an alternate embodiment, the ligation template may be a chimeric RNA-DNA oligonucleotide. In another embodiment, the ligation template may be a DNA oligonucleotide. In yet another embodiment, the ligation template may be a DNA oligonucleotide comprising at least one PCR blocker, such that the ligation template may not serve as a primer for PCR amplification. Non-limiting examples of a PCR blocker include a dideoxynucleotide, an amine group, a methyl group, a phosphate group, or carbon spacers.

The ligation template may comprise standard, nonstandard, modified, or derivatized nucleotides, or nucleotide analogs as detailed above for the 5′ linker. In particular, a nucleotide may be derivatized with biotin, digoxigenin, a fluorophore, or a magnetic particle. Phosphodiester bonds or phosphothioate bonds may link the nucleotides of the ligation template.

As described above for the 5′ linker, the ligation template may be free in solution, or it may be attached to a solid support. In the later case, the 5′ linker and the mature small RNA may hybridize to the immobilized ligation template and be ligated while indirectly attached to the solid support. The ligation product may then be released from the solid support for analysis.

The term “hybridization,” as used herein, refers to the process of hydrogen bonding, annealing, or base pairing between two single-stranded nucleic acids. The “stringency” of hybridization is typically determined by the conditions of temperature and ionic strength. Nucleic acid hybrid stability is generally expressed as the melting temperature or Tm, which is the temperature at which the hybrid is 50% denatured under defined conditions. Equations have been derived to estimate the Tm of a given hybrid; the equations take into account the G+C content of the nucleic acid, the nature of the hybrid (e.g., DNA:DNA, DNA:RNA, etc.), the length of the nucleic acid probe, etc. (e.g., Sambrook et al. (1989) Molecular Cloning: A Laboratory Manual, Cold Spring Harbor Press, Cold Spring Harbor, N.Y., chapter 9). To maximize the rate of annealing between the probe and its target, hybridizations are generally carried out in solutions of high ionic strength (6×SSC or 6×SSPE) at a temperature that is about 20-25° C. below the Tm. If the sequences to be hybridized are not exact complements, then the hybridization temperature is reduced from about 1° C. to about 1.5° C. for every 1% of mismatch. After hybridization, the unbound probe is removed by washing under conditions that are as stringent as possible (i.e., with solutions of low ionic strength at a temperature about 12-20° C. below the calculated Tm). As an example, stringent conditions typically involve hybridizing at 68° C. in 6×SSC/5×Denhardt's solution/1.0% SDS and washing in 0.2×SSC/0.1% SDS at 65° C. The optimal hybridization conditions generally differ between hybridizations performed in solution and hybridizations using immobilized nucleic acids. One skilled in the art will appreciate which parameters to manipulate to optimize hybridization.

In a preferred embodiment, the complementary ligation template is an RNA oligonucleotide that is about 14-16 nucleotides in length, having about 8-10 nucleotides at the 5′ end having complementarity to the mature small RNA and about 6-8 nucleotides at the 3′ end having complementarity to the 5′ linker.

(iii) Ligase

Ligation of double-stranded nucleic acids is generally faster and more efficient than ligation of single-stranded nucleic acids. Unidirectional (end-specific) ligation is also highly desirable. Thus, ligation of the 5′ linker to the 5′ end of the mature small RNA is performed in the presence of a complementary ligation template. The ligation will be catalyzed by a template-dependent DNA ligase. A template-dependent DNA ligase is generally defined as either a ligase that only catalyzes bond formation in a duplex nucleic acid or a ligase that functions more efficiently in a duplex nucleic acid. The template-dependent DNA ligase may catalyze the formation of phosphodiester bonds in DNA or RNA. Additionally, the template-dependent DNA ligase may be a ligase that requires ATP as a cofactor. Examples of a suitable ATP-dependent, template-dependent DNA ligase include, but are not limited to, T4 DNA ligase, vaccinia DNA ligase, and a mammalian DNA ligase.

In a preferred embodiment, the ligase is T4 DNA ligase. The concentration of T4 DNA ligase may range from about 1 Weiss unit to about 30 Weiss units per 10 μl reaction mixture, and more preferably from about 5 Weiss units to about 10 Weiss units per 10 μl-reaction mixture. T4 DNA ligase is an ATP dependent ligase. The amount of ATP in the ligation reaction mixture may range from 1 nM to about 10 mM, and more preferably from about 1 μM to about 1 mM.

The conditions of the ligation reaction are typically adjusted so that the ligase functions near its optimal activity level. The pH utilized during the ligation reaction may range from about 6.5 to about 8.5, more preferably from about 7.0 to about 8.0, and most preferably from about 7.6 to about 7.8. A buffering agent may be utilized to adjust and maintain the pH at the desired level. Representative examples of suitable buffering agents include MOPS, HEPES, TAPS, Bicine, Tricine, TES, PIPES, MES, sodium acetate or a Tris buffer. In a preferred embodiment, the buffer may be Tris-HCl and the pH level may be about 7.8.

The ligation reaction mixture may further comprise a divalent cation. A suitable divalent cation includes calcium, magnesium, or manganese. In a preferred embodiment, the divalent salt may be magnesium chloride, manganese chloride, or a combination thereof. The concentration of the divalent cation may range from about 0.2 mM to about 15 mM, and more preferably from about 1 mM to about 5 mM.

The reaction mixture may further comprise a reducing agent. Non-limiting examples of suitable reducing agents include dithiothreitol and β-mercaptoethanol. A ribonuclease (RNase) inhibitor may also be added to the ligation reaction mixture. The RNase inhibitor may be a naturally occurring protein or it may be a recombinant protein. The reaction mixture may also comprise a clouding agent to facilitate the hybridization of the nucleic acids. Examples of suitable clouding agents include polyethylene glycol (PEG) 4000 and PEG 8000.

The temperature of the ligation reaction is typically adjusted so that the ligase functions near its optimal level. The temperature of the ligation reaction may range from about 14° C. to about 40° C. In a preferred embodiment, the ligation reaction is performed at about 37° C. The duration of the reaction is generally sufficient to allow completion of the reaction at a given temperature. For reactions conducted at 37° C., the duration of the reaction may range from about 0.5 hour to about 18 hours, and more preferably from about 1 hour to about 3 hours.

In a preferred embodiment, the ligation reaction is performed in the presence of about 100 ng of isolated RNA, 50 nM of 5′ linker, 50 nM of ligation template, 10 Weiss units of T4 DNA ligase, 40 mM Tris-HCl (pH 7.8), 3 mM MgCl₂, 100 μM ATP, 100 μM DTT, 5% PEG 4000, and 2 units RNase inhibitor at about 37° C. for about 2 hours.

(c) Assaying the Ligation Product

The method further comprises assaying the ligation product comprising the 5′ linker and the mature small RNA, such that the mature small RNA is detected. The assay may be quantitative, such that the amount or mass of the mature small RNA in a sample may be determined. Alternatively, the assay may be qualitative, such that the presence of a mature small RNA may be determined in the sample, but its level may not be measured. Furthermore, the assay may be such that the mature small RNA may be isolated from the sample for further study.

An amplification method may be used to assay the ligation product. Non-limiting examples of suitable amplification methods include quantitative real-time PCR, quantitative end-point PCR, and standard PCR.

To amplify the ligation product of this invention, the ligation product is generally converted into a DNA copy. In one embodiment, a DNA copy of the ligation product may be synthesized during PCR by using a reverse primer that hybridizes to the 3′ end of the ligation product, whereby a thermostable DNA polymerase extends the primer using the ligation product as the template. The reverse primer may be complementary to the small RNA portion of the ligation product, or the reverse primer may span the ligation junction and hybridize with both the small RNA and the linker portions. Alternatively, the reverse primer may hybridize with a region at the 3′ end of the small RNA portion of the ligation product, such that at least one ribonucleotide at the 5′ end of the small RNA is not paired with the primer. On skilled in the art will appreciate that the number of unpaired nucleotides at the 5′ end of the small RNA can and will vary depending on the application and the type of DNA polymerase used. In an alternate embodiment, the ligation product may be converted into a DNA copy by the action of a reverse transcriptase and a reverse transcriptase primer that is complementary to a region at the 3′ end of the ligation product. The reverse transcriptase may be MMLV, AMV, or a variant thereof.

The reverse primer used to generate a DNA copy of the ligation product will generally also be used to amplify the product, in conjunction with a forward primer. In general, the forward primer corresponds to a sequence of the 5′ linker, such that the forward primer may hybridize with a DNA copy of the ligation product. The sequence of the 5′ linker that corresponds to the forward primer can and will vary, depending upon, for example, the desired length of the amplified fragment. Both of the forward and reverse PCR primers may comprise standard, nonstandard, derivatized, and modified nucleotides, as detailed above. In one embodiment, the forward and reverse primers may each comprise at least one modified nucleotide, such as a locked nucleic acid (LNA). In general, nucleic acids comprising a LNA have increased thermal stability and hybridization specificity. In a preferred embodiment, the reverse primer comprises at least two LNAs. The forward and reverse primers may each range from about 18 nucleotides to about 24 nucleotides in length.

Quantitative real-time PCR (qPCR) may be used to assay the ligation product. During this method, the amount of PCR product is followed cycle-by-cycle in real time. To measure the amount of PCR product, the reaction may be performed in the presence of a fluorescent dye whose fluorescence increases greatly when bound to double-stranded DNA. Non-limiting examples of suitable fluorescent dyes include SYBR® Green I (Molecular Probes/Invitrogen, Carlsbad, Calif.), Pico Green I (Molecular Probes/Invitrogen, Carlsbad, Calif.), EvaGreen™ (Biotium, Inc., Hayward, Calif.), ethidium bromide, and acridine orange. The reaction may also be performed with a fluorogenic reporter probe that is specific for the DNA being amplified. Non-limiting examples of reporter probes include TaqMan® (Applied Biosystems, Foster City, Calif.), Molecular Beacons (Tyagi and Kramer (1996) Nature Biotechnology 14:303-308), and Scorpion® primers (Whitcombe D. et al. (1999) Nature Biotechnology 17:804-807). The aforementioned probes depend on Förster Resonance Energy Transfer (FRET) to quench the fluorescence signal via the coupling of a fluorogenic dye molecule and a quencher moiety on the same or different oligonucleotide substrates. The fluorescence signal is generated when the fluorogenic dye molecule and the quencher are decoupled via enzymatic or physical means. Fluorescence values are generally recorded during each cycle and represent the amount of product amplified to that point in the amplification reaction. The cycle during which the fluorescence exceeds a defined threshold value is defined as the threshold cycle (Ct). In general, the amount of starting material may be calculated by determining the Ct value of the sample and comparing it to Ct values of control samples.

Quantitative end-point PCR (Willey, J. C. et al. (1998) Am. J. Resp. Cell Mol. Biol. 19: 6-17) may also be used to assay the ligation product. This method is similar to qPCR in that the reaction is generally performed in the presence of a fluorescent dye or a fluorogenic probe/primer, but the amount of PCR product is not followed cycle-by-cycle. Rather the PCR product is analyzed at the end of the reaction by resolving the amplified product by electrophoresis on a DNA chip, an agarose gel, or a capillary, and then measuring the fluorescence of the product. The reaction typically includes a co-amplified internal control or a co-amplified synthetic nucleic acid for sample normalization.

A standard PCR method may also be used to assay the ligation product. Standard PCR procedures are well known in the art and information regarding these procedures may be found in Ausubel et al. (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y.

A hybridization method may also be used to assay the ligation product. Non-limiting examples of suitable hybridization methods include nucleic acid microarray, nucleic acid-coupled microsphere array platform, and branched DNA technology.

Microarray analyses may be performed using commercially available equipment, such as the GenChip® technology (Affymetrix, Santa Clara, Calif.) or the Microarray System (Incyte, Fremont, Calif.), and following the manufacturer's protocols. Typically, single-stranded nucleic acids are attached (arrayed) to a microchip surface. The arrayed sequences are then hybridized (probed) with nucleic acids, which may be fluorescently labeled. After stringent washing to remove the non-specifically bound nucleic acids, the chip surface is generally scanned by confocal laser microscopy or by another detection method, such as a CCD camera. Methods of analysis of the raw fluorescent data are known in the art. A variety of arrayed nucleic acid and probe combinations may be used to detect the ligation product of this invention. The 5′ linker, a sequence complementary to the 5′ linker, or a sequence complementary to the small RNA may be attached to the chip surface. The ligation product may be formed in silico on the chip surface, or the ligation product may be formed in solution (and later attached to the chip). The ligation product, a probe complementary to the ligation product, a probe complementary to the 5′ linker, or a probe complementary to the small RNA may be hybridized to the nucleic acids attached to the chip surface. The 5′ linker, a probe complementary to the 5′ linker, the ligation template, the small RNA, a probe complementary to the small RNA, or combinations thereof may be fluorescently labeled.

The ligation product may also be assayed using a microsphere array platform, such as the microbeads from Luminex. These microscopic polystyrene beads are internally color-coded with fluorescent dyes, such that each bead has a unique spectral signature (of which there are up to 100). Beads with the same signature are generally tagged with a specific oligonucleotide that may bind the target nucleic acid. The target, in turn, is also tagged with a fluorescent reporter. Hence, there are two sources of color, one from the microbead and the other from the reporter molecule on the target. The beads are then incubated with the sample containing the target. The small size/surface area of the beads and the three dimensional exposure of the beads to the target allows for nearly solution-phase kinetics during the binding reaction. The captured targets are then detected by high-tech fluidics based upon flow cytometry in which lasers excite the internal dyes that identify each bead and also any reporter dye captured during the assay. As detailed in the preceding paragraph, a variety of combinations of nucleic acids may be attached to the microbeads or used to probe the microbeads.

For some assays, it may be necessary to remove the ligation template from the ligation product before the ligation product is assayed. For most PCR amplification methods, this may not be necessary because the duplexed ligation template and ligation product are generally separated during the denaturation step of a PCR cycle. Alternatively, the ligation template may be removed from the ligation product by the exonuclease activity of some DNA polymerases used in PCR. For some of the hybridization-based methods, however, it may be necessary to degrade or remove the ligation template from the ligation product before assaying the ligation product. To accomplish this, the ligation template may further comprise a biotin tag to be used to capture the ligation product/ligation template complex by its interaction with streptavidin. The ligation template may then be released from the ligation product by heat or chemical means. In another embodiment, the ligation template may further comprise a magnetic particle, which may be used to separate the ligation template from the ligation product. In yet another embodiment, an exonuclease may be used to selectively degrade a ligation template.

Lastly, the amplified products may be used for subsequent analyses. Non-limiting examples include molecular cloning, DNA sequencing, PCR amplifications, hybridization assays, membrane hybridizations, microarray assays, microbead array platforms, electrophoretic assays, transfections, transformations, and microinjections.

(d) Detecting a Plurality of Mature Small RNAs

The above-described method for the detection of a single mature small RNA may be modified for multiplex detection of a plurality of mature small RNAs whose sequences are known. For this, the 5′ linker is ligated to a plurality of mature small RNAs in the presence of a plurality of ligation templates, whereby a plurality of ligation products may be formed in a single ligation reaction. Each ligation template in the plurality of ligation templates comprises a distinct 5′ region that hybridizes with the 5′ end of a specific mature small RNA, and each ligation template has a constant 3′ region that hybridizes with the 3′ end of the 5′ linker. As demonstrated in Example 10, seven distinct mature small RNAs were detected in a single multiplex reaction using the method of the invention.

The methods of assaying the ligation product described above may be readily modified to assay a plurality of ligation products. In one embodiment, the plurality of ligation products may be assayed individually. For example, a first aliquot of the ligation mixture may be PCR amplified using a forward primer that corresponds to the 5′ linker and a first reverse primer that is complementary to the 5′ end of a first small RNA; a second aliquot of the ligation mixture may be PCR amplified using the forward primer and a second reverse primer that is complementary to the 5′ end of a second small RNA, etc. Likewise, hybridization methods may be performed sequentially to probe for specific small RNAs. In an alternate embodiment, the plurality of ligation products may be assayed simultaneously via a multiplex assay. For example, multiplex PCR may be performed using reverse primers having different fluorophores or gene-specific probes having different fluorophores. Alternatively, microarray assays may be devised to detect more than one target, and multiplexing Luminex microsphere arrays may be set up for as many as 100 different targets.

### (II) Method for Detecting a Population of Mature Small RNAs

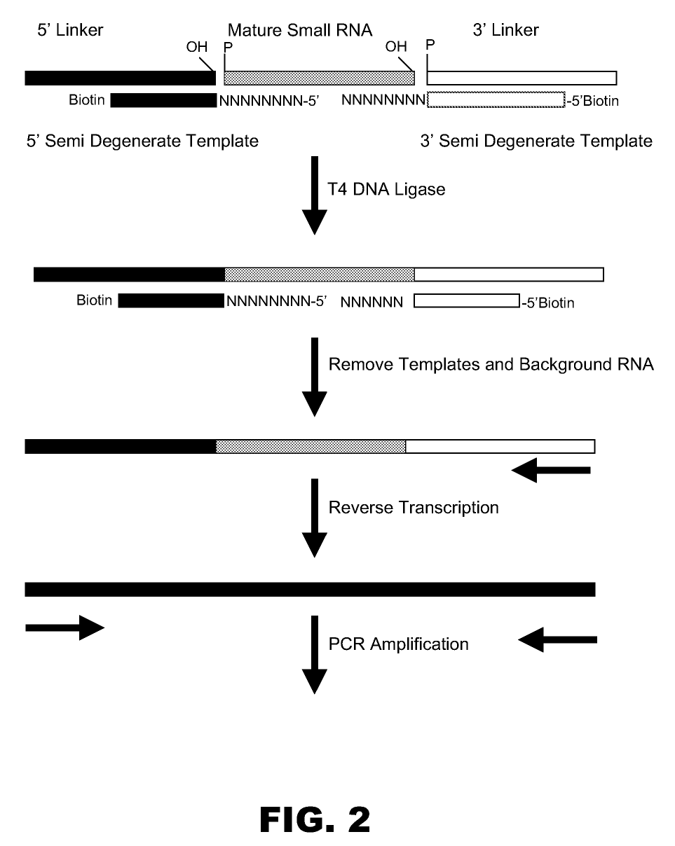

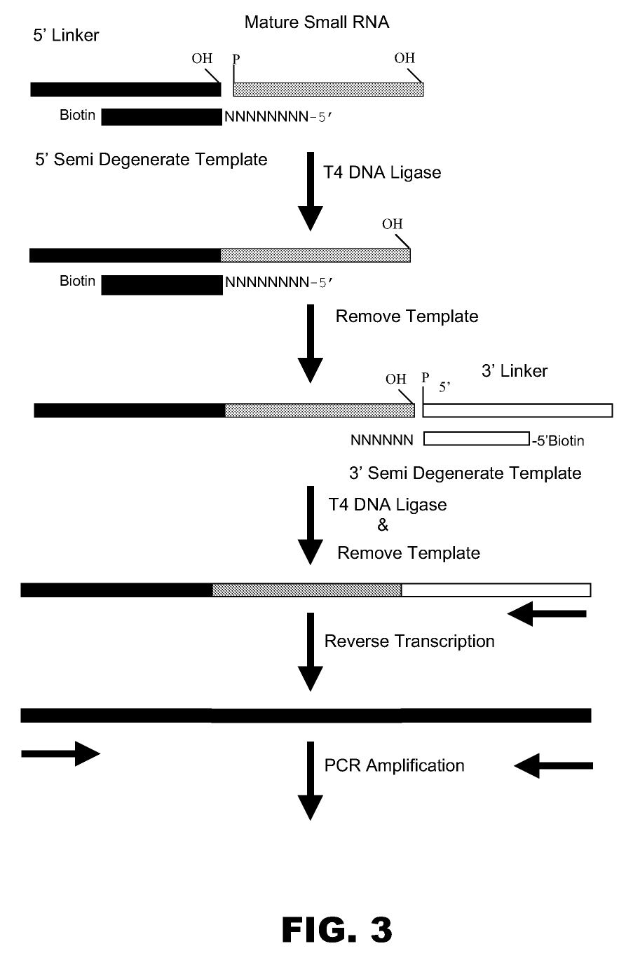

A further aspect of the invention is the provision of a method for detecting a population of mature small RNAs whose sequences may or may not be known. The method comprises two ligation steps that may be performed simultaneously (see FIG. 2) or sequentially (see FIG. 3). In the first ligation step, a first linker is attached to one end of a mature small RNA in a population of mature small RNAs in the presence of a first semi-degenerate ligation template that spans the first ligation junction, such that a plurality of first ligation products is formed. In the second ligation step, a second linker is attached to the mature small RNA end of a ligation product in the plurality of first ligation products in the presence of a second semi-degenerate ligation template that spans the second ligation junction, such that a plurality of second ligation products is formed. The method further comprises assaying the plurality of second ligation products, whereby a population of mature small RNAs is detected.

The first step of the method comprises providing a sample comprising a population of mature small RNAs. The sample may be derived from animals, plants, fungi, protists, viruses, bacteria, or archaea. The sample may be an isolated preparation of small RNA, an isolated preparation of total RNA, a crude cellular extract, or an intact cell. Total RNA or small RNA may be isolated from cells or cellular extracts using commercially available kits or techniques well known in the art. The population of mature small RNAs may comprise miRNAs, siRNAs, rasiRNAs, tasiRNAs, piRNAs, 21-U RNAs, or combinations thereof, as detailed above in section (I)(a).

This method comprises attaching a first linker and a second linker to each end of a mature small RNA, which has a 5′ terminal phosphate and a 3′ terminal hydroxyl group. The first linker may be a 5′ linker and the second linker may be a 3′ linker, or vice versa. The linkers may comprise ribonucleotides, deoxyribonucleotides, or a combination thereof. Other features of the linkers were described above in section (I)(b)(i). A 3′ linker is similar to a 5′ linker, except that a 3′ linker has a 5′ terminal phosphate. Furthermore, in embodiments in which the 3′ linker may be a chimeric DNA-RNA polynucleotide, the ribonucleotide(s) is at the 5′ end. In embodiments in which the 3′ linker may form a stem-loop structure, the stem comprises the 3′ end of the linker and the 5′ end of the linker is single-stranded. Additionally, in embodiments in which the 3′ linker may further comprise a RNA polymerase promoter site, the promoter site is in the reverse orientation.

Ligation of the first and second linkers to the ends of a mature small RNA is preformed in the presence of first and second semi-degenerate ligation templates, each of which is complementary to one of the ligation junctions. The first semi-degenerate ligation template may be a 5′ semi-degenerate ligation template, and the second semi-degenerate ligation template may be a 3′ semi-degenerate ligation template, or vice versa. Each of the semi-degenerate ligation templates comprises a constant region that hybridizes with one of the linkers; i.e., the constant region of the 5′ semi-degenerate ligation template is complementary to the 3′ end region of a 5′ linker, and the constant region of the 3′ semi-degenerate ligation template is complementary to the 5′ end region of a 3′ linker. Each of the semi-degenerate ligation templates further comprises a degenerate region that may hybridize with a specific mature small RNA in the population of mature small RNAs. The degenerate region of each semi-degenerate ligation template comprises a random mix of nucleotides, such that thousands of different nucleotide combinations are represented.

The first linker and the first semi-degenerate ligation template are designed to work together. Thus, if the first linker is a 5′ linker, then the first semi-degenerate ligation template is a 5′ semi-degenerate ligation template, etc. Likewise, the second linker and the second semi-degenerate ligation template are designed to work together. Thus, if the second linker is a 3′ linker, then the second semi-degenerate ligation template is a 3′ semi-degenerate ligation template, etc.

The semi-degenerate ligation templates may comprise ribonucleotides, deoxyribonucleotides, or a combination thereof. The semi-degenerate ligation templates may comprise standard, nonstandard, modified, or derivatized nucleotides, or nucleotide analogs as detailed above. In particular, a nucleotide may be derivatized with a fluorophore, biotin, digoxigenin, or a magnetic particle. Phosphodiester or phosphothioate bonds may link the nucleotides or nucleotide analogs of the semi-degenerate ligation templates. The semi-degenerate ligation templates can and will vary in length, as detailed above in section (I)(b)(ii) for the complementary ligation templates. A typical semi-degenerate ligation template may comprise ribonucleotides and be about 14-16 nucleotides in length with about 6-8 nucleotides that hybridize with a mature small RNA and about 8-10 nucleotides that hybridize with one of the linkers.

A 5′ semi-degenerate ligation template comprises a 3′ region that hybridizes under stringent conditions with the 3′ end of the 5′ linker, and a degenerate 5′ region comprising a random mix of nucleotides, such that each template may hybridize with a discrete mature small RNA in the population of mature small RNAs. One skilled in the art will appreciate that the number of nucleotides comprising the degenerate region determines the number of possible template combinations, and hence, the number of mature small RNAs that may be hybridized. The 5′ semi-degenerate ligation template may also be designed such that the nucleotide in the position of the template that corresponds to the 5′ terminus of the small RNA is not entirely random. In general, U is the most common nucleotide at the 5′ terminus of all known mature microRNAs. U is also the invariable nucleotide at the 5′ terminus of mature 21 U-RNAs. Thus, the ligation template may comprise an A in the position that corresponds to the 5′ terminus of the small RNA. The nucleotide in that position in the ligation template may be altered to target small RNAs that have an A, a C, or a G at the 5′ terminus. Thus, 5′ semi-degenerate ligation templates may be combined in a ratio in accordance with the expected frequency of small RNAs with a specific nucleotide at the 5′ terminus. Example 11 demonstrated that a 5′ semi-degenerate ligation template directed the ligation of several different microRNAs to a 5′ linker.

A 3′ semi-degenerate ligation template comprises a 5′ region that hybridizes under stringent conditions with the 5′ end of the 3′ linker, and a degenerate 3′ region comprising a random mix of nucleotides, such that each template may hybridize with a discrete mature small RNA in the population of mature small RNAs. One skilled in the art will appreciate that the number of nucleotides comprising the degenerate region determines the number of possible template combinations, and hence, the number of small RNAs that may be hybridized.

Ligation of the first and second linkers to the population small RNAs may be performed simultaneously, and the ligation products may be separated from the ligation reaction materials before the second ligation products are assayed, as described in Example 12 and diagrammed in FIG. 2. For example, each of the semi-degenerate ligation templates may further comprise a biotin tag. The interaction between biotin and streptavidin may be used to capture the ligation product/ligation template complexes, and the ligation products may be released from the immobilized ligation templates by heat or chemical means. In another embodiment, each of the semi-degenerate ligation templates may further comprise a magnetic particle, which may be used to separate the ligation templates from the ligation products.

Alternatively, the first and second linkers may be ligated to the small RNAs sequentially, and the ligation products may be separated from the reaction materials after each ligation reaction, as described in Example 13 and diagrammed in FIG. 3. For this, the first linker is ligated to one end of the population of small RNAs to form first ligation products, the first ligation products are separated from the reaction materials using one of the methods described above, the second linker is ligated to the other end of the population of small RNAs to form second ligation products, and the second ligation products are separated from the reaction materials using one of the methods described above.

The method further comprises assaying the second ligation products by converting them into DNA copies and then amplifying the copies. The second ligation products may be converted into DNA copies by the action of a reverse transcriptase in conjunction with a reverse primer that is complementary to a region of the 3′ linker. The reverse transcriptase may be MMLV, AMV, or a variant thereof. The DNA copies of the second ligation products may be amplified with any of the PCR methods described above in section (I)(c) using a forward primer that corresponds to a sequence of the 5′ linker and a reverse primer that is complementary to a sequence of the 3′ linker. Alternatively, the DNA copies of the second ligation products may be amplified by in vitro transcription using at least one RNA polymerase, provided that each linker further comprises a corresponding RNA polymerase promoter sequence. The RNA polymerase may be T3, T7, or SP6 RNA polymerase. The two linkers may each have promoter sequence sites for the same RNA polymerase or different RNA polymerases.

Lastly, the amplified products may be used for subsequent analyses. Non-limiting examples include molecular cloning, DNA sequencing, PCR amplifications, hybridization assays, membrane hybridizations, microarray assays, microbead array platforms, electrophoretic assays, transfections, transformations, and microinjections.

### (III) Kits for Detecting Mature Small RNAs

A further aspect of the invention encompasses kits to detect mature small RNAs. Provided is a kit for detecting a mature small RNA whose sequence is known. In one embodiment, the kit comprises a 5′ linker to be ligated to the 5′ end of the mature small RNA, a complementary ligation template that spans the ligation junction between the 5′ linker and the mature small RNA, a ligase, and instructions for using the kit. The 5′ linker, ligation template, and ligase were described above in section (I)(b). In another embodiment, the kit further comprises a forward PCR primer that corresponds to a sequence of the 5′ linker, a reverse PCR primer that is complementary to the 5′ end sequence of the small RNA, and a set of reagents for quantitative PCR. The quantitative PCR may be real-time or end-point. In an alternate embodiment, the kit further comprises a plurality of complementary ligation templates, rather than a single ligation template, such that a plurality of mature small RNAs, whose sequences are known, may be detected. The 5′ end of each complementary ligation template in the plurality of complementary ligation templates is complementary to a discrete mature small RNA in the plurality of mature small RNAs.

Also provided is a kit for detecting a population of mature small RNAs whose sequences are known or unknown. In one embodiment, the kit comprises a 5′ linker, a 5′ semi-degenerate ligation template, a 3′ linker, a 3′ semi-degenerate ligation template, a ligase, and instructions for using the kit. The linkers and semi-degenerate templates were described above in section (II). In another embodiment, the kit further comprises a reverse transcriptase and a reverse primer that is complementary to a region of the 3′ linker. In yet another embodiment, the kit further comprises a forward PCR primer that corresponds to a sequence of the 5′ linker, a reverse PCR primer that is complementary to a sequence of the 3′ linker, and a set of reagents for quantitative PCR. The quantitative PCR may be real-time or end-point.

### DEFINITIONS

The term “mature small RNA,” as used herein, refers to a small RNA molecule generally comprising about 20-30 nucleotides that was processed from a larger RNA precursor. A mature small RNA has a 5′ terminal phosphate and a 3′ terminal hydroxyl group.

The term “ligation product,” as used herein, refers to a hybrid molecule comprising at least one linker and a mature small RNA.

The terms “nucleic acid,” “oligonucleotide,” and “polynucleotide,” as used herein, refer to sequences of linked nucleotides. The nucleotides may be deoxyribonucleotides or ribonucleotides. In general, oligonucleotides comprise few nucleotides, e.g., less than about 50, whereas polynucleotides comprise many nucleotides.

As various changes could be made in the above composition, products and methods without departing from the scope of the invention, it is intended that all matter contained in the above description and in the examples given below, shall be interpreted as illustrative and not in a limiting sense.

### EXAMPLES

The following examples illustrate various embodiments of the invention.

### Example 1

### Detection of Phosphorylated MicroRNAs Using a Template-Dependent Ligation Method

An experiment was designed to test whether a phosphorylated small RNA could be distinguished from a non-phosphorylated small RNA using a template-dependent ligation and PCR detection method. As depicted in FIG. 1, the method comprised ligating a 5′ linker to the 5′ end of a phosphorylated small RNA in the presence of a complementary ligation template that spanned the ligation junction. The ligation template was complementary to the 5′ end of the small RNA and the 3′ end of the 5′ linker. The ligation was catalyzed by T4 DNA ligase, a template-dependent ligase. The ligated linker/small RNA chimera was then directly amplified and quantitated using real-time PCR (qPCR) using a forward primer that corresponded to a portion of the 5′ linker and a reverse primer that was complementary to the small RNA. The small RNA that was detected and quantified was the human let-7a microRNA (hsa-let-7a) (see Table 1).

The oligonucleotides needed for this experiment (see Table 1) were synthesized by conventional techniques. Phosphorylated and non-phosphorylated hsa-let-7a microRNAs (SEQ ID NOs:1, 2) were made, with the 5′ phosphate in the phosphorylated form incorporated during synthesis. The 5′ linker (SEQ ID NO:3) was a DNA-RNA hybrid, i.e., the DNA polynucleotide was modified with a ribonucleotide at the 3′ terminus. At a temperature below 50° C., the 5′ linker formed an 8-bp stem (underlined), a 69-base loop between the complementary sequences in the stem, and an 8 nucleotide 3′ overhang, which served as a ligation arm.

TABLE 1
Oligonucleotides Used for Detecting a Phosphorylated microRNA.

| | | |
|---|---|---|
| Name | Sequence (5′ to 3′)* | SEQ ID NO: |

| | | |
|---|---|---|
| Phosphorylated hsa- | pUGAGGUAGUAGGUUGUAUAGUU | 1 |
| let-7a microRNA | | |
| Non-phosphorylated | UGAGGUAGUAGGUUGUAUAGUU | 2 |
| hsa-let-7a microRNA | | |
| 5′ Linker 1 | ggcaggtaatacgactcactataggtcgagagtcagggag | 3 |
| | cactccagctgcgaccaggagaggtgtctcagcagagtac | |
| | ctgccagcaactG | |
| Ligation template 1 | UACUACCUCACAGUUGCU | 4 |
| Ligation template 2 | CGUGCUGCUACAGUUGCU | 5 |
| Reverse primer 1 | aactatacaacctactacctcac | 6 |
| Forward primer | ggcaggtaatacgactcacta | 7 |

*Ribonucleotides are shown in uppercase, and deoxyribonucleotides are in lowercase.

Two ligation templates were synthesized—one that was complementary to the microRNA and the 5′ linker (SEQ ID NO:4) and one that was not complementary to this microRNA but was complementary to the 5′ linker (SEQ ID NO:5). Both were RNA oligonucleotides. The 8 nucleotides at the 3′ end region of each were complementary to the 3′ overhang of the 5′ linker. The 10 nucleotides at the 5′ end region of ligation template 1 were complementary to the 5′ end region of the hsa-let-7a microRNA. The 10 nucleotides at the 5′ end region of ligation template 2 were not complementary to this microRNA.

Forward and reverse PCR primers (SEQ ID NOs:6, 7) were also synthesized. Twenty-two of the 23 nucleotides of reverse primer 1 were complementary to the hsa-let-7 microRNA, and the (23^{rd}) nucleotide at the 3′ end of the primer was complementary to the ribonucleotide at the 3′ terminus of the 5′ inker. The forward primer corresponded to the 5′ end region of the 5′ linker.

Each ligation test was conducted in a 10 μl reaction containing 40 mM Tris-HCl (pH 7.8), 0.2 mM MgCl₂, 10 μM ATP, 100 μM DTT, 5% PEG 4000, 2 units RNase inhibitor, 15 Weiss units T4 DNA ligase, 50 nM 5′ linker, 50 nM complementary or non-complementary ligation template, 100 μM 5′ phosphorylated hsa-let-7a microRNA or non-phosphorylated hsa-let-7a microRNA. Reactions that contained no 5′ linker, no ligation template, no microRNA, or no T4 DNA ligase were also evaluated concurrently. All reactions were incubated at 37° C. on a thermal cycler for 2 hours. Following ligation, 1 μl of each reaction was mixed with 12.5 μl 2×SYBR Green JumpStart Taq ReadyMix (Sigma, Product Code S4438; Sigma-Aldrich, St. Louis, Mo.), 0.25 μl 100×ROX, as internal reference dye, (Sigma, Product Code R4526), 0.5 μl of 20 μM reverse primer 1, 0.5 μl of 20 μM forward primer, and 10.25 μl of water. Each ligation reaction was assayed in triplicate. Amplification and detection was conducted on a Mx3000P Real-Time PCR System (Stratagene; La Jolla, Calif.) with the following thermal profile: 1 cycle: 94° C. for 2 minutes; 40 cycles: 94° C. for 15 seconds, 55° C. for 30 seconds, 72° C. for 30 seconds, and 84° C. for 30 seconds for fluorescent data collection. Amplification plots and dissociation curves were generated with a standard thermal profile.

The results are summarized in Table 2 as the mean and standard deviation of cycle threshold (Ct) values. The Ct value represents the PCR cycle during which the fluorescence exceeded a defined threshold level. This experiment confirmed that this ligation-dependent method was able to specifically detect a 5′ phosphorylated microRNA and did not detect a non-phosphorylated microRNA. Furthermore, this experiment revealed that a 5′ linker, a complementary ligation template, and a ligase were required.

TABLE 2
Detection of a Phosohorylated microRNA

| | T4 DNA | Ligation | hsa-let-7a | Ct Value |
|---|---|---|---|---|
| 5′ Linker | Ligase | Template | microRNA | (mean ± sd) |
| Yes | Yes | Complementary | Phosphorylated | 23.51 ± 0.14 |
| Yes | Yes | Complementary | Non- | No Ct |
| | | | phosphorylated | |
| Yes | Yes | Complementary | No microRNA | No Ct |
| Yes | Yes | Non- | Phosphorylated | No Ct |
| | | complementary | | |
| Yes | Yes | No template | Phosphorylated | No Ct |
| No | Yes | Complementary | Phosphorylated | No Ct |
| Yes | No | Complementary | Phosphorylated | No Ct |

Note:
No Ct represents no detectable PCR products after 40 cycles.

### Example 2

### Optimization of Reverse Primer Design

This small RNA detection method is based upon PCR amplification of the ligation product. Because the ligation product comprises RNA, which is not “read” efficiently by a “natural” DNA polymerase, the design of the reverse primer is important. The reverse primer may hybridize to the microRNA and prime DNA synthesis starting from the 3′ end of the (DNA) linker. To determine whether the reverse primer needs to overlap the junction between the small RNA and the linker, three different reverse primers were designed and tested. Reverse primer 2 (SEQ ID NO:9) had the same overall design as the reverse primer used in Example 1; i.e., it was complementary to the microRNA and had an “overlapping” nucleotide at the 3′ end that was complementary to the ribonucleotide at the 3′ terminus of the 5′ linker. Reverse primer 3 (SEQ ID NO:10) was complementary to the microRNA and comprised two locked nucleic acid (LNA) modifications (indicated by the + sign upstream of a modified nucleotide), but it lacked the overlapping nucleotide at the 3′ terminus. Reverse primer 4 (SEQ ID NO:11) was complementary to the microRNA but it contained neither modified nucleotides nor an overlapping nucleotide.

The reverse primers were tested for their ability to amplify mmu-mir-16 microRNA in a population of small RNAs isolated from mouse. The sequence of mmu-mir-16 microRNA (SEQ ID NO:8) is identical between human and mouse and is presented in Table 3, along with the primer sequences. The 5′ linker was SEQ ID NO:3, and the complementary ligation template was SEQ ID NO:5, both of which are presented in Table 3.

TABLE 3′
Oligonucleotides Used to Test Reverse Primer Design.

| | | |
|---|---|---|
| Name | Sequence (5′ to 3′)* | SEQ ID NO: |

| | | |
|---|---|---|
| mmu-mir-16 microRNA | UAGQAGCACGUAAAUAUUGGCG | 8 |
| 5′ Linker 1 | ggcaggtaatacgactcactataggtcgagagtcagggagcactc | 3 |
| | cagctgcgaccaggagaggtgtctcagcagagtacctgccagca | |
| | | actG |
| Ligation template 2 | CGUGCUGCUACAGUUGGU | 5 |
| Reverse primer 2 | cgccaatatttacgtgctgctac | 9 |
| Reverse primer 3 | cgccaa+gtatt+gtacgtgctgcta | 10 |
| Reverse primer 4 | cgccaatatttacgtgctgcta | 11 |
| Forward primer | ggcaggtaatacgactcacta | 7 |

*Ribonucleotides are shown in uppercase, and deoxyribonucleotides are in lowercase.

Small RNA was isolated from mouse liver tissue using a small RNA purification kit (Sigma, Product Code SNC10). Each ligation was conducted in a 20 μl reaction containing 40 mM Tris-HCl (pH 7.8), 0.2 mM MgCl₂, 10 μM ATP, 100 μM DTT, 5% PEG 4000, 4 units RNase inhibitor, 30 Weiss units T4 DNA ligase, 50 nM 5′ linker, 50 nM ligation template, and 200 ng of the isolated small RNA. Corresponding ligation reactions containing no RNA or no T4 DNA ligase were also conducted. All ligation reactions were incubated at 37° C. on a thermal cycler for 2 hours. The SYBR Green qPCR amplification and detection method was essentially the same as in Example 1. Each primer set was tested in duplicate for each ligation product.

The results of this experiment are presented in Table 4, as the mean and standard deviation of Ct values. This experiment revealed that reverse primers 2 and 3 were comparable to each other, but were more efficient than reverse primer 4, in PCR amplification. These data indicate that a reverse primer does not have to overlap the ligation junction, as long as it contains modified nucleotides, such as LNAs. Thus, the reverse primer may be designed in a variety of different ways for efficient PCR amplification. Furthermore, this example again illustrated that only ligated products were amplified.

TABLE 4
Evaluation of Reverse Primers

| | | Ct Value |
|---|---|---|
| Ligation Reaction | Reverse Primer | (mean ± sd) |
| Mouse liver small RNA | Reverse primer 2 | 23.23 ± 0.28 |
| | Reverse primer 3 | 23.73 ± 0.35 |
| | Reverse primer 4 | 25.26 ± 0.13 |
| No RNA | Reverse primer 2 | No Ct |
| | Reverse primer 3 | No Ct |
| | Reverse primer 4 | No Ct |
| No T4 DNA ligase | Reverse primer 2 | No Ct |
| | Reverse primer 3 | No Ct |
| | Reverse primer 4 | No Ct |

Note:
No Ct represents no detectable PCR products after 40 cycles.

### Example 3

### Optimization of 5′ Linker Design

An experiment was designed to determine whether a ribonucleotide at the 3′ terminus of the 5′ linker increased the efficiency of microRNA detection. For this, 5′ linker 2 was synthesized (SEQ ID NO:12) whose sequence was identical to the 5′ linker used in Examples 1 and 2 but without the ribose modification at the 3′ terminus. Table 5 presents the oligonucleotides used in this experiment. The mmu-mir-16 microRNA (SEQ ID NO:8) was detected in RNA isolated from mouse using one of the two 5′ linkers, with the ligation reactions performed in the presence of different levels of MgCl₂ and MnCl₂.

TABLE 5
Oligonucleotides Used to Test 5′ Linker Modifications.

| | | |
|---|---|---|
| Name | Sequence (5′ to 3′)* | SEQ ID NO: |

| | | |
|---|---|---|
| mmu-mir-16 | UAGCAGCACGUAAAUAUUGGCG | 8 |
| microRNA | | |
| 5′ Linker 1 | ggcaggtaataogactcactataggtcgagagtcagggagcactc | 3 |
| (with 3′1 ribose) | cagctgcgaccaggagaggtgtctcagcagagtacctgccagca | |
| | actG | |
| 5′ Linker 2 | ggcaggtaatacgactcactataggtcgagagtcagggagcactc | 12 |
| (with no ribose) | cagctgcgaccaggagaggtgtctcagcagagtacctgccagca | |
| | actg | |
| Ligation template 2 | CGUGCUGCUACAGUUGCU | 5 |
| Reverse primer 2 | cgccaatatttacgtgctgctac | 9 |
| Forward primer | ggcaggtaatacgactcacta | 7 |

*Ribonucleotides are shown in uppercase, and deoxyribonucleotides are in lowercase.

Each ligation was conducted in a 10 μl reaction containing 40 mM Tris-HCl (pH 7.8), 100 μM ATP, 100 μM DTT, 5% PEG 4000, 2 units RNase inhibitor, 15 Weiss units T4 DNA ligase, 50 nM of one of the two 5′ linkers, 50 nM of ligation template, 100 ng of mouse liver small RNA sample, and a given level of divalent cation. A ligation master mix was prepared and aliquots of the mix were added into individual reaction tubes containing different levels of MgCl₂ and/or MnCl₂. The ligation reactions were incubated at 37° C. for 2 hours on a thermal cycler. Following ligation, 1 μl of each ligation reaction was mixed with 12.5 μl of 2×SYBR Green JumpStart Taq ReadyMix (Sigma, Product Code S4438), 0.25 μl of 100×ROX, as internal reference dye, (Sigma, Product Code R4526), 0.5 μl of 20 μM forward primer, 0.5 μl of 20 μl reverse primer, and 10.25 μL of water. Each ligation reaction was tested in duplicate. The qPCR thermal profile was as follows: 1 cycle: 94° C. for 2 minutes; 40 cycles: 94° C. for 15 seconds, 60° C. for 1 minute, and 84° C. for 30 seconds for fluorescent data collection. Amplification plots and dissociation curves were generated with a standard thermal profile.

The results are summarized in Table 6. The results showed that a single ribose modification at the 3′ terminus of the 5′ linker increased ligation efficiency to various degrees depending upon the levels of divalent ion. The difference between the modified and the non-modified 5′ linkers was small (about 1 Ct), however, at high levels of MgCl₂ (10 mM MgCl₂, and 10 mM MgCl₂+1 mM MnCl₂). These data revealed that both ribose modified and non-modified 5′ linkers can be used to detect a phosphorylated small RNA.

TABLE 6
Effect of 5′ Linker Modification.

| | 5′ Linker | Ct Value | Ct |
|---|---|---|---|
| Divalent Ion in Ligation | Modification | (mean ± sd) | Difference |
| 10 mM MgCl₂ | 3′-Ribose | 21.98 ± 0.00 | 1.03 |
| | None | 23.01 ± 0.04 | |
| 5 mM MgCl₂ | 3′-Ribose | 21.78 ± 0.04 | 2.26 |
| | None | 24.04 ± 0.07 | |
| 2.5 mM MgCl₂ | 3′-Ribose | 21.41 ± 0.17 | 1.65 |
| | None | 23.06 ± 0.06 | |
| 2 mM MgCl₂ | 3′-Ribose | 21.61 ± 0.01 | 2.82 |
| | None | 24.43 ± 0.10 | |
| 10 mM MgCl₂ + 1 mM MnCl₂ | 3′-Ribose | 21.87 ± 0.04 | 1.21 |
| | None | 23.08 ± 0.19 | |
| 5 mM MgCl₂ + 1 mM MnCl₂ | 3′-Ribose | 21.63 ± 0.12 | 1.29 |
| | None | 22.92 ± 0.05 | |
| 2.5 mM MgCl₂ + 1 mM MnCl₂ | 3′-Ribose | 21.50 ± 0.05 | 1.62 |
| | None | 23.12 ± 0.02 | |
| 1 mM MgCl₂ + 1 mM MnCl₂ | 3′-Ribose | 21.35 ± 0.08 | 2.47 |
| | None | 23.82 ± 0.06 | |
| 1 mM MnCl₂ | 3′-Ribose | 21.48 ± 0.04 | 4.12 |
| | None | 25.64 ± 0.21 | |
| No divalent ion | 3^{′}-Ribose | 33.77 ± 0.33 | 1.22 |
| | None | 34.99 ± 0.27 | |

### Example 4

### Optimization of 5′ Linker and Ligation Template Configuration

To test the optimal configuration of the 5′ linker and the ligation template, four different 5′ linkers were synthesized in which all had the same stem-loop sequence and ribose modification as SEQ ID NO:3, but each of which had a different length of 3′ overhang, ranging from 6 to 12 nucleotides. The four 5′ linkers were paired with 6 different complementary ligation templates, which ranged from 14 to 24 nucleotides in length. The 3′ region of each ligation template was complementary to the 3′ overhang of the 5′ linker and the rest of each ligation template was complementary to the 5′ end region of the micro RNA (mmu-mir-16 microRNA). Table 7 presents the six 5′ linker and ligation template pairs, with the 3′ overhangs of the 5′ linkers underlined. The mmu-mir-16 microRNA (SEQ ID NO:8) was detected in RNA isolated from mouse.

TABLE 7
5′ Linker and Ligation Template Combinations

| | |
|---|---|
| Sequence (5′ to 3′)* | SEQ ID NO: |

| | | | |
|---|---|---|---|
| Pair 1 | 5′ Linker 3 | ggcaggtaatacgactcactataggtcgagagtcag | 13 |
| | | ggagcactccagctgcgaccaggagaggtgtctca | |
| | | gcagagtacctgcccaatgA | |
| | Ligation template 3 | UGCUGCUAUCAUUG | 14 |
| Pair 2 | 5′ Linker 3 | see above | 13 |
| | Ligation template 4 | CGUGCUGCUAUCAUUG | 15 |
| Pair 3 | 5′ Linker 4 | ggcaggtaatacgactcactataggtcgagagtcag | 16 |
| | | ggagcactccagctgcgaccaggagaggtgtctca | |
| | | gcagagtacctgccagcaatgA | |
| | Ligation template 5 | UGCUGCUAUCAUUGCU | 17 |
| Pair 4 | 5′ Linker 4 | see above | 16 |
| | Ligation template 6 | CGUGCUGCUAUCAUUGCU | 18 |
| Pair 5 | 5′ Linker 5 | ggcaggtaatacgactcactataggtcgagagtcag | 19 |
| | | ggagcactccagctgcgaccaggagaggtgtctca | |
| | | gcagagtacctgccagagcaatctA | |
| | Ligation template 7 | CGUGCUGCUAUCAUUGCUCU | 20 |
| Pair 6 | 5′ Linker 6 | ggcaggtaatacgactcactataggtcgagagtcag | 21 |
| | | ggagcactccagctgcgaccaggagaggtgtctca | |
| | | gcag agtacctgcctcagagcaatgA | |
| | Ligation template 8 | UACGUGCUGCUAUCAUUGCUCUGA | 22 |

*Ribonucleotides are shown in uppercase, and deoxyribonucleotides are in lowercase.

Samples of small RNA and total RNA were isolated from mouse liver tissue essentially as described in Example 2. Each ligation was performed in a 10 μl reaction containing 40 mM Tris-HCl (pH 7.8), 1.5 mM MgCl₂, 100 μM ATP, 100 μM DTT, 5% PEG 4000, 2 units RNase inhibitor, 15 Weiss units T4 DNA ligase, 50 nM 5′ linker, 50 nM ligation template, and 100 ng of small RNA or total RNA. Corresponding reactions containing no RNA were also run. The reactions were incubated at 37° C. for 2 hours in a thermal cycler. The SYBR Green qPCR amplification and detection method was essentially the same as described in Example 3. The forward primer was SEQ ID NO:7 and the reverse primer was SEQ ID NO:10.

The results are summarized in Table 8. In general, the longer the overhang and the longer the ligation template, the lower the Ct value. Thus, various configurations of 5′ linker and ligation template can be used to detect a 5′ phosphorylated small RNA.

TABLE 8
Detection of microRNA Using Different 5′ Linker
and Ligation Template Combinations

| | Length of 3′ | Length of | | |
|---|---|---|---|---|
| | overhang on | ligation | | Ct Value |
| Pair | 5′ linker | template | RNA Sample | (mean ± sd) |
| 1 | 6 | 14 | Small RNA | 22.90 ± 0.10 |
| | | | Total RNA | 28.48 ± 0.06 |
| | | | No RNA | No Ct |
| 2 | 6 | 16 | Small RNA | 22.69 ± 0.12 |
| | | | Total RNA | 29.45 ± 0.02 |
| | | | No RNA | 36.95 ± 0.83 |
| 3 | 8 | 16 | Small RNA | 21.08 ± 0.13 |
| | | | Total RNA | 28.22 ± 0.04 |
| | | | No RNA | 33.65 ± 0.18 |
| 4 | 8 | 18 | Small RNA | 21.38 ± 0.00 |
| | | | Total RNA | 28.06 ± 0.07 |
| | | | No RNA | 33.52 ± 0.14 |
| 5 | 10 | 20 | Small RNA | 21.05 ± 0.00 |
| | | | Total RNA | 27.78 ± 0.03 |
| | | | No RNA | No Ct |
| 6 | 12 | 24 | Small RNA | 19.97 ± 0.04 |
| | | | Total RNA | 26.79 ± 0.09 |
| | | | No RNA | No Ct |

Note:
No Ct represents no detectable PCR products after 40 cycles.

### Example 5

### Optimization of Level of ATP in the Ligation Reaction

An experiment was conducted to evaluate different levels of ATP in the ligation reaction used to detect a phosphorylated small RNA. The mmu-mir-16 microRNA (SEQ ID NO:8) was detected in RNA isolated from mouse. A different 5′ linker (SEQ ID NO:23) having a ribonucleotide at the 3′ end of the 3′ overhang (underlined) was used (see Table 9). The ligation template (SEQ ID NO:24) was complementary to the junction of the microRNA and the 5′ linker.

TABLE 9
Oligonucleotides Used to Optimize the Level of ATP.

| | | |
|---|---|---|
| Name | Sequence (5′ tc 3′)* | SEQ ID NO: |

| | | |
|---|---|---|
| mmu-mir-16 | UAGCAGCACGUAAAUAUUGGCG | 8 |
| micro RNA | | |
| 5′ Linker 7 | ggcaggtaatacgactcactataggtcgagagtcagggagcactcc | 23 |
| | agctgcgaccaggagaggtgtctcagcagagtacctgcccaacaG | |
| Ligation template 9 | UGCUGCUACUGUUG | 24 |
| Reverse primer 2 | cgccaatatttacgtgctgctac | 9 |
| Reverse primer 3 | cgccaa+gtatt+acgtgctgcta | 10 |
| Forward primer | ggcaggtaatacgactcacta | 7 |

*Ribonucleotides are shown in uppercase, and deoxyribonucleotides are in lowercase.

Samples of small RNA and total RNA were isolated from mouse liver as described previously. Each ligation, with a different level of ATP, was conducted in a 10 μl reaction containing 40 mM Tris-HCl (pH 7.8), 1.5 mM MgCl₂, 100 μM DTT, 5% PEG 4000, 2 units RNase inhibitor, 50 nM 5′ linker, 50 nM ligation template, 100 ng of isolated RNA, and 15 Weiss units T4 DNA ligase. Two ligation reaction master mixes, one containing small RNA and the other total RNA, were prepared and aliquots of the mixes were added into individual reaction tubes containing different levels of ATP. All of the ligation reactions were incubated at 37° C. for 2 hours. The SYBR Green qPCR amplification and detection method was essentially the same as described in Example 3. Two different reverse primers (SEQ ID NO:9 or SEQ ID NO:10) were used in combination with a forward primer (SEQ ID NO:7). Each ligation reaction was tested in duplicate per pair of primers.

The Ct values are shown in Table 10. The results revealed that the optimal ATP level was between about 1 μM and about 1 mM. The results also showed that the ligation reactions comprising small RNA proceeded moderately well, even when no ATP was added to the reactions. Without being limited to any particular theory, this suggests that the T4 DNA ligase was purified with the ADP cofactor bound to the enzyme active center and, thus, was capable of catalyzing a certain level of ligation when the target microRNA was present at a relatively high level, as in the small RNA preparation. Thus, this experiment revealed that the ligation reaction can be carried out over a wide range of ATP concentrations.

TABLE 10
Optimization of ATP Levels.

| | | Ct Value (mean ± sd) | | | |
|---|---|---|---|---|---|
| | | Reverse Primer 2 | | Reverse Primer 3 | |
| | | (SEQ ID NO: 9) | | (SEQ ID NO: 10) | |
| ATP Level | | Small RNA | Total RNA | Small RNA | Total RNA |
| 10 | mM | 31.84 ± 0.04 | 34.48 ± 0.33 | 34.11 ± 0.43 | No Ct |
| 1 | mM | 19.23 ± 0.02 | 25.44 ± 0.44 | 21.80 ± 0.22 | 28.36 ± 0.10 |
| 0.1 | mM | 17.75 ± 0.00 | 24.29 ± 0.10 | 20.37 ± 0.11 | 26.72 ± 0.09 |
| 10 | μM | 18.30 ± 0.06 | 24.36 ± 0.18 | 20.46 ± 0.13 | 26.88 ± 0.11 |
| 1 | μM | 19.30 ± 0.02 | 26.12 ± 0.42 | 21.45 ± 0.03 | 27.97 ± 0.17 |
| 0.1 | μM | 21.13 ± 0.01 | 28.03 ± 0.04 | 23.20 ± 0.01 | 30.03 ± 0.02 |
| 10 | nM | 22.29 ± 0.08 | 31.30 ± 0.19 | 24.38 ± 0.02 | 32.82 ± 0.02 |
| 1 | nM | 22.72 ± 0.00 | 33.28 ± 0.13 | 24.92 ± 0.08 | 35.05 & No Ct |

| | | | | |
|---|---|---|---|---|
| No ATP | 22.71 ± 0.06 | 33.96 ± 0.10 | 25.29 ± 0.23 | 33.85 & No Ct |

Note:
No Ct represents no detectable PCR products after 40 cycles.

### Example 6

### Optimization of Ligation Buffer and pH

To determine the optimal buffer composition and pH for the ligation reactions, seven different 10× ligation buffers were prepared and evaluated for the detection of a phosphorylated small RNA. The compositions of the 10× buffers were as follows:

Buffer 1: 400 mM Sodium Acetate, pH 7.0, 20 mM MgCl₂,1 mM ATP, 1 mM DTT

Buffer 2: 400 mM Tris-HCl, pH 7.0, 20 mM MgCl₂, 1 mM ATP, 1 mM DTT

Buffer 3: 400 mM Tris-HCl, pH 7.2, 20 mM MgCl₂, 1 mM ATP, 1 mM DTT

Buffer 4: 400 mM Tris-HCl, pH 7.4, 20 mM MgCl₂, 1 mM ATP, 1 mM DTT

Buffer 5: 400 mM Tris-HCl, pH 7.6, 20 mM MgCl₂, 1 mM ATP, 1 mM DTT

Buffer 6: 400 mM Tris-HCl, pH 7.8, 20 mM MgCl₂, 1 mM ATP, 1 mM DTT

Buffer 7: 400 mM Tris-HCl, pH 8.0, 20 mM MgCl₂, 1 mM ATP, 1 mM DTT

The mmu-mir-16 microRNA (SEQ ID NO:8) was detected in RNA isolated from mouse. The 5′ linker was SEQ ID NO:23 and the ligation template was SEQ ID NO:24. Preparations of small RNA and total RNA sample were prepared from mouse liver as described previously. Each ligation was conducted in a 10 μl reaction containing 1× of one of the seven 10× Buffers (or water only), 5% PEG 4000, 2 units RNase inhibitor, 15 Weiss units T4 DNA ligase, 50 nM 5′ linker, 50 nM ligation template, and 100 ng of RNA. Two reaction master mixes without a 10× buffer were prepared; one comprising small RNA and the other comprising total RNA. Aliquots of the mixes were added into individual reaction tubes containing different buffers. The reactions were incubated at 37° C. for 2 hours. The SYBR Green qPCR amplification and detection method was essentially the same as described in Example 3. The forward primer was SEQ ID NO:7, and the reverse primer was SEQ ID NO:9. Each ligation reaction was tested in duplicate.

The results are shown in Table 11. This experiment demonstrated that a buffer was required during the ligation reaction, but that the pH of the buffer can range from about pH 7.0 to about pH 8.0.

TABLE 11
Effect of Buffer and pH During Ligation.

Ct Value (mean ± sd)

| Buffer and pH | Small RNA | Total RNA |
|---|---|---|
| Sodium Acetate, pH 7.0 | 18.79 ± 0.06 | 25.13 ± 0.02 |
| Tris-HCl, pH 7.0 | 18.49 ± 0.01 | 24.93 ± 0.00 |
| Tris-HCl, pH 7.2 | 18.33 ± 0.10 | 24.83 ± 0.01 |
| Tris-HCl, pH 7.4 | 18.27 ± 0.16 | 24.61 ± 0.13 |
| Tris-HCl, pH 7.6 | 18.13 ± 0.04 | 24.68 ± 0.00 |
| Tris-HCl, pH 7.8 | 18.51 ± 0.05 | 24.90 ± 0.21 |
| Tris-HCl, pH 8.0 | 18.59 ± 0.08 | 24.85 ± 0.06 |
| No Buffer | No Ct | No Ct |

Note:
No Ct represents no detectable PCR products after 40 cycles.

### Example 7

### Optimization of the Level of T4 DNA Ligase

Different amounts of T4 DNA ligase were evaluated in the ligation reaction used to detect a phosphorylated small RNA. The mmu-mir-16 microRNA (SEQ ID NO: 8) was detected in RNA isolated from mouse. Two different pairs of 5′ linker and ligation template were used for this evaluation. Pair 1 comprised SEQ ID NO:23 as the 5′ linker and SEQ ID NO:24 as the ligation template. Pair 2 comprised SEQ ID NO:21 as the 5′ linker and SEQ ID NO:22 as the ligation template. Small RNA was isolated from mouse liver as described previously. Ligation reactions were conducted in 10 μl reactions containing 40 mM Tris-HCl (pH 7.6), 2 mM MgCl₂, 100 μM ATP, 100 μM DTT, 5% PEG 4000, 2 units RNase inhibitor, 50 nM 5′ linker, 50 nM ligation template, 100 ng of small RNA, and a given amount of T4 DNA ligase. Each enzyme level was tested in duplicate with each 51 linker/ligation template pair. The reactions were incubated at 37° C. for 2 hour. The SYBR Green qPCR amplification and detection method was essentially the same as used in Example 3. The forward primer was SEQ ID NO:7 and the reverse primer was SEQ ID NO:10. Each reaction was performed in duplicate.

The Ct values are presented in Table 12. This experiment revealed that low levels of T4 DNA ligase were as effective as high levels of T4 DNA ligase. A subsequent experiment was conducted to evaluate even lower amounts of the enzyme with the first pair of linker/template. The results are presented in the lower part of Table 12, and again confirmed that a wide range of ligase concentrations can be used to detect a phosphorylated small RNA.

TABLE 12
Optimization of Ligase Levels.

| | Length of 3′ | Length of | T4 DNA ligase | |
|---|---|---|---|---|
| | overhang on | ligation | (Weiss unit/μL | Ct Value |
| Pair | 5′ linker | template | reaction) | (mean ± sd) |
| 1 | 6 | 14 | No ligase | No Ct |
| | | | 0.5 | 21.53 ± 0.09 |
| | | | 1.0 | 21.38 ± 0.08 |
| | | | 1.5 | 21.69 ± 0.14 |
| | | | 2.0 | 21.86 ± 0.05 |
| | | | 2.5 | 22.35 ± 0.17 |
| | | | 3.0 | 22.18 ± 0.15 |
| 2 | 12 | 24 | No ligase | No Ct |
| | | | 0.5 | 21.80 ± 0.09 |
| | | | 1.0 | 21.70 ± 0.09 |
| | | | 1.5 | 21.88 ± 0.10 |
| | | | 2.0 | 22.38 ± 0.40 |
| | | | 2.5 | 22.18 ± 0.15 |
| | | | 3.0 | 22.24 ± 0.14 |
| 1 | 6 | 14 | 0.125 | 22.86 ± 0.32 |
| | | | 0.25 | 22.27 ± 0.32 |
| | | | 0.5 | 21.65 ± 0.04 |
| | | | 0.75 | 21.87 ± 0.08 |
| | | | 1.5 | 21.82 ± 0.12 |

Note:
No Ct represents no detectable PCR products after 40 cycles.

### Example 8

### Optimization of the Ligation Time

An experiment was conducted to evaluate the optimal duration of the ligation reaction used to detect a phosphorylated small RNA. The mmu-mir-16 microRNA (SEQ ID NO:8) was detected in RNA isolated from mouse. The 5′ linker used was SEQ ID NO:23, and the ligation template was SEQ ID NO:24. Small RNA and total RNA were isolated from mouse liver tissues as described previously. A preparation of total RNA was also prepared from Arabidopsis leaf tissues with the same purification kit as described in Example 2 and was used as a background RNA control. Each ligation reaction was conducted in 10 μl reaction containing 40 mM Tris-HCl (pH 7.6), 3 mM MgCl₂, 100 μM ATP, 100 μM DTT, 5% PEG 4000, 2 units RNase inhibitor, 50 nM 5′ linker, 50 nM ligation template, 100 ng of an RNA sample, 10 Weiss units of T4 DNA ligase. Three ligation master mixes, each containing a different RNA sample, were prepared and aliquots of each mix were added into individual reaction tubes. The reaction tubes were incubated at 37° C., and tubes were removed at one-hour intervals during the course of 8 hr and stored at −20° C. The SYBR Green qPCR amplification and detection method was essentially the same as described in Example 3. Each ligation reaction was tested in duplicate. The forward primer was SEQ ID NO:7, and the reverse prime r was SEQ ID NO: 10.

The results are presented in Table 13. This experiment revealed that there was only a small decrease in Ct value over the 8-hour ligation period. It also showed that there was little change in the background signal from the Arabidopsis RNA sample over the same time course. This example revealed that the ligation reaction proceeded quite rapidly and can be allowed to proceed for different periods of time.

TABLE 13
Effect of Ligation Time on the Detection of microRNA.

| | Ct Value (mean ± sd) | | |
|---|---|---|---|
| | Mouse Liver | Mouse Liver | |
| Ligation | Small | Total | Arabidopsis Leaf |
| Time (hr) | RNA Sample | RNA Sample | Total RNA Sample |
| 1 | 22.29 ± 0.10 | 28.98 ± 0.04 | 38.41 and No Ct |
| 2 | 22.02 ± 0.06 | 28.06 ± 0.12 | 38.61 and No Ct |
| 3 | 21.59 ± 0.09 | 27.66 ± 0.08 | 38.50 and No Ct |
| 4 | 21.38 ± 0.19 | 27.28 ± 0.18 | 38.13 and No Ct |
| 5 | 21.23 ± 0.01 | 27.23 ± 0.05 | 37.69 and No Ct |
| 6 | 21.19 ± 0.01 | 26.97 ± 0.09 | 39.21 ± 1.05 |
| 7 | 21.06 ± 0.04 | 26.98 ± 0.07 | 38.61 ± 0.30 |
| 8 | 21.46 ± 0.06 | 27.14 ± 0.08 | 39.33 and No Ct |

Note:
No Ct represents no detectable PCR products after 40 cycles.

### Example 9

### Optimization of the Amount of RNA in the Ligation Reaction

An experiment was conducted to evaluate different amounts of RNA in the ligation reaction used to detect a phosphorylated small RNA. The mmu-mir-16 microRNA (SEQ ID NO:8) was detected in RNA isolated from mouse. The 5′ linker was SEQ ID NO:23, and the ligation template was SEQ ID NO:24. Preparations of small RNA and total RNA were prepared from mouse liver tissues as described previously. Ligations were conducted in 10 μl reactions containing 40 mM Tris-HCl (pH 7.6), 3 mM MgCl₂, 100 μM ATP, 100 μM DTT, 5% PEG 4000, 2 units RNase inhibitor, 50 nM 5′ linker, 50 nM ligation template, 10 Weiss units of T4 DNA ligase, and a given amount of one of the two RNA samples. A master mix was prepared and aliquots of the mix were added into individual tubes containing different amounts of RNA. All reactions were incubated at 37° C. for 2 hours. The SYBR Green QPCR amplification and detection method was essentially the same as described in Example 3. Each ligation reaction was tested in duplicate. The forward primer was SEQ ID NO:7, and the reverse primer was SEQ ID NO:10.

The Ct values are presented in Table 14. This experiment revealed that when the level of small RNA ranged from 50 to 300 ng, the Ct value was reduced about one unit as the amount of RNA was doubled, indicating a linear relationship. A linear relationship was also maintained between Ct and RNA level when the level of total RNA ranged from about 100 to 400 ng. This example showed that widely different amounts of starting RNA can be used to detect a phosphorylated small RNA.

TABLE 14
Effect of Different Levels of RNA During Ligation.

| | |
|---|---|
| Mouse Liver Small RNA | Mouse Liver Total RNA |

| Amount | Ct Value | Amount | Ct Value |
|---|---|---|---|
| (ng) | (mean ± sd) | (ng) | (mean ± sd) |
| 50 | 22.96 ± 0.15 | 100 | 27.49 ± 0.01 |
| 100 | 22.00 ± 0.03 | 200 | 26.89 ± 0.14 |
| 150 | 21.20 ± 0.11 | 300 | 26.06 ± 0.01 |
| 200 | 21.11 ± 0.07 | 400 | 25.81 ± 0.00 |
| 250 | 20.59 ± 0.04 | 500 | 25.63 ± 0.01 |
| 300 | 20.43 ± 0.05 | 600 | 25.57 ± 0.09 |
| 350 | 20.14 ± 0.07 | 700 | 25.48 ± 0.01 |
| 400 | 20.45 ± 0.08 | 800 | 25.72 ± 0.01 |

### Example 10

### Single Versus Multiplex MicroRNA Ligation

This experiment was designed to compare the detection of a plurality of microRNAs using individual ligation reactions versus a single multiplex ligation reaction. Seven different microRNAs were detected in HeLa cells, and six different microRNAs were detected in Arabidopsis leaf tissues. Small RNA was prepared from HeLa adherent cells and Arabidopsis leaf tissues respectively, using a small RNA isolation kit as described previously. The microRNA sequences and corresponding ligation template sequences for each human microRNA and each Arabidopsis microRNA are presented in Tables 15 and 16, respectively. The 5′ linker for each reaction was SEQ ID NO:23. Each single microRNA ligation was conducted in a 10 μl reaction containing 40 mM Tris-HCl (pH 7.6), 3 mM MgCl₂, 100 μM ATP, 100 μM DTT, 5% PEG 4000, 2 units RNase inhibitor, 50 nM 5′ linker, 50 nM ligation template, 10 Weiss units of T4 DNA ligase, and 100 ng RNA sample. Each multiplex microRNA ligation was conducted in a 50 μl reaction containing 40 mM Tris-HCl (pH 7.6), 3 mM MgCl₂, 100 μM ATP, 100 μM DTT, 5% PEG 4000, 10 units RNase inhibitor, 50 nM 5′ linker probe, 10 nM of each ligation template, 50 Weiss units of T4 DNA ligase, and 500 ng RNA sample. The multiplex microRNA ligation for HeLa RNA sample comprised 7 different ligation templates, each targeting a specific human microRNA. The multiplex microRNA ligation for Arabidopsis RNA sample comprised 6 different ligation templates, each targeting a specific Arabidopsis microRNA. All reactions were incubated at 37° C. for 2 hours. The SYBR Green qPCR amplification and detection method was essentially the same as described in Example 3. Each amplification reaction comprised 1 μl of ligation reaction; each ligation reaction was run in triplicate. The forward primer was SEQ ID NO:7. Reverse primers, specific for each microRNA, are presented in Tables 15 and 16.

The amplification results are presented in Tables 15 and 16. There were no significant differences between the Ct values of single ligated microRNAs and the Ct values of multiplex ligated microRNAs. This example illustrated that a plurality of microRNAs can be ligated to the same 5′ linker in a single ligation reaction, allowing for multiplex detection of several different microRNAs in a sample.

TABLE 15
Single vs. Multiplex Ligation Human microRNAs

| | |
|---|---|
| MicroRNA, Ligation Template | Ct Value |
| and Reverse Primer | (mean ± sd) |

| | | SEQ | Single | Multiplex |
|---|---|---|---|---|
| microRNA | Sequence (5′ to 3′)* | ID NO. | Ligation | Ligation |
| hsa-mir-21: | UAGCUUAUCAGACUGAUGUUGA | 25 | 18.57 ± 0.14 | 18.34 ± 0.10 |
| Lig. Temp. 10: | AUAAGCUACUGUUG | 26 | | |
| Rev. Primer 5: | tcaacatcagtctgataagctac | 27 | | |
| hsa-mir-1: | UGGAAUGUAAAGAAGUAUGUA | 28 | 32.03 ± 0.15 | 31.77 ± 0.14 |
| Lig. Temp. 11: | ACAUUCCACUGUUG | 29 | | |
| Rev. Primer 6: | tacatacttctttacattccac | 30 | | |
| hsa-mir-330: | GCAAAGCACACGGCCUGCAGAGA | 31 | 31.05 ± 0.21 | 30.17 ± 0.14 |
| Lig. Temp. 12: | UGCUUUGCCUGUUG | 32 | | |
| Rev. Primer 7: | tctctgcaggccgtgtgctttgcc | 33 | | |
| hsa-mir-23B: | AUCACAUUGCCAGGGAUUACC | 34 | 20.23 ± 0.04 | 19.89 ± 0.08 |
| Lig. Temp. 13: | CAAUGUGAUCUGUUG | 35 | | |
| Rev. Primer 8: | ggtaatccctggcaatgtgatc | 36 | | |
| hsa-mir-130A: | CAGUGCAAUGUUAAAAGGGCAU | 37 | 25.82 ± 0.12 | 25.92 ± 0.08 |
| Lig. Temp. 14: | UUGCACUGCUCUUG | 38 | | |
| Rev. Primer 9: | atgcccttttaacattgcactgc | 39 | | |
| hsa-let-7a: | UGAGGUAGUAGGUUGUAUAGUU | 2 | 23.16 ± 0.10 | 22.89 ± 0.05 |
| Lig. Temp. 15: | CUACCUCACUGUUG | 40 | | |
| Rev. Primer 1: | aactatacaacctactacctcac | 6 | | |
| hsa-mir-16: | UAGCAGCACGUAAAUAUUGGCG | 8 | 21.99 ± 0.10 | 21.74 ± 0.06 |
| Lig. Temp. 9: | UGCUGCUACUGUUG | 24 | | |
| Rev. Primer 2: | cgccaatatttacgtgctgctac | 9 | | |

*Ribonucleatides are shown in uppercase, and deoxyribonucleotides are in lowercase.

TABLE 16
Single vs. Multiplex Ligation Arabidopsis microRNAs

| | |
|---|---|
| MicroRNA, Ligation Template | Ct Value |
| and Reverse Primer | (mean ± sd) |

| | | SEQ | Single | Multiplex |
|---|---|---|---|---|
| microRNA | Sequence (5′ to 3′)* | ID NO. | Ligation | Ligation |
| ath-miR156a | UGACAGAAGAGAGUGAGCAC | 41 | 26.97 ± 0.02 | 27.60 ± 0.08 |
| Lig. Temp. 16: | UUCUGUCACUGUUG | 42 | | |
| Rev. Primer 10: | gtgctcactctcttctgtcac | 43 | | |
| ath-miR172a: | AGAAUCUUGAUGAUGCUGCAU | 44 | 18.14 ± 0.08 | 19.21 ± 0.12 |
| Lig. Temp. 17: | CAAGAUUCUCUGUUG | 45 | | |
| Rev. Primer 11: | atgcagcatcatcaagattctc | 46 | | |
| ath-miR169a: | CAGCCAAGGAUGACUUGCCGA | 47 | 21.79 ± 0.11 | 23.02 ± 0.12 |
| Lig. Temp. 18: | CUUGGCUGCUGUUG | 48 | | |
| Rev. Primer 12: | tcggcaagtcatccttggctgc | 49 | | |
| ath-miR159a: | UUUGGAUUGAAGGGAGCUCUA | 50 | 19.68 ± 0.06 | 20.28 ± 0.03 |
| Lig. Temp. 19: | CAAUCCAAACUGUUG | 51 | | |
| Rev. Primer 13: | tagagctcccttcaatccaaac | 52 | | |
| ath-miR170: | UGAUUGAGCCGUGUCAAUAUC | 53 | 26.28 ± 0.03 | 27.85 ± 0.13 |
| Lig. Temp. 20: | CUCAAUCACUGUUG | 54 | | |
| Rev. Primer 14: | gatattgacacggctcaatcac | 55 | | |
| ath-miR166a: | UCGGACCAGGCUUCAUUCCCC | 56 | 28.11 ± 0.04 | 28.30 ± 0.18 |
| Lig. Temp. 21: | UGGUCCGACUGUUG | 57 | | |
| Rev. Primer 15: | ggggaatgaagcctggtccgac | 58 | | |

*Ribonucleotides are shown in uppercase, and deoxyribonucleotides are in lowercase.

### Example 11

### Ligation with a Semi-Degenerate Template

An experiment was conducted to determine whether a semi-degenerate template could be used for the ligation of several different microRNAs in a single ligation reaction. A semi-degenerate ligation template was synthesized with the following sequence (5′-3′): NNNNNNNACUGUUG (SEQ ID NO: 59), wherein each N represents A, U, G, or C. The semi-degenerate template represented 16,384 template combinations. The 6 nucleotides at the 3′ end of the template were complementary to the 6 nucleotides at the 3′ end of the 5′ linker. The A at the 7^{th} position from the 3′ end of the template was designed to target microRNAs that have a U at the 5′-most position, since this is the most common nucleotide at the 5′ end of all known microRNAs. The A at that position in the template may be substituted with U, G, or C for targeting microRNAs that have an A, a C or a G at the 5′-most position, respectively. Thus, to target all possible microRNAs, the four different semi-degenerate templates may be combined in a ratio in accordance with the expected frequency of U, A, G, C occurring at the 5′-most position of the target microRNAs. The microRNAs detected In this experiment were hsa-let-7a (SEQ ID NO:2), hsa-mir-16 (SEQ ID NO:8), and hsa-mir-21 SEQ ID NO:25). The 5′ linker was SEQ ID NO: 23.

Small RNA was prepared from HeLa adherent cells as described previously. Each ligation was conducted in a 20 νl reaction containing 40 mM Tris-HCl (pH 7.6), 3 mM MgCl₂, 100 μM ATP, 100 μM DTT, 5% PEG 4000, 4 units RNase inhibitor, 100 nM 5′ linker, 1 mM ligation template, 20 Weiss units of T4 DNA ligase, and 200 ng RNA sample. Ligation reactions comprising no ligation template were also conducted concurrently. Ligation reactions were incubated at 18° C. or 16° C. for 14 hours.

The SYBR Green qPCR amplification and detection method was essentially the same as described in Example 3. Each amplification reaction comprised 1 μl of ligation reaction; reactions were performed in duplicate. The forward primer was SEQ ID NO:7. The reverse primers were SEQ ID NO:6, 9, and 27, which were used to detect hsa-let-7a, hsa-mir-16, and hsa-mir-21, respectively.

The amplification results are shown in Table 17. This experiment revealed that a semi-degenerate ligation template could be used for the ligation and subsequent detection of several different microRNAs in a single ligation reaction.

TABLE 17
Semi-Degenerate Template Detects Multiple MicroRNAs

| | Semi-degenerate | Ct Value (mean ± sd) | |
|---|---|---|---|
| microRNA | Template | Ligation at 18° C. | Ligation at 16° C. |
| hsa-let-7a | Yes | 26.10 ± 0.14 | 26.34 ± 0.02 |
| | No | 34.31 ± 0.07 | 34.52 ± 0.28 |
| hsa-mir-16 | Yes | 23.16 ± 0.08 | 23.27 ± 0.06 |
| | No | 33.93 ± 0.18 | 34.59 ± 0.63 |
| hsa-mir-21 | Yes | 19.58 ± 0.01 | 19.55 ± 0.04 |
| | No | 33.35 ± 0.11 | 34.23 ± 0.72 |

### Example 12

### Genome-Wide Detection of Mature Small RNAs—One-Step Ligation

Semi-degenerate ligation templates will be used to detect a population of known or unknown mature small RNAs in a sample. As diagramed in FIG. 2, this method comprises ligating a 5′ linker to the 5′ end of a phosphorylated mature small RNA through the use of a 5′ semi-degenerate ligation template that spans the 5′ ligation junction. The 5′ semi-degenerate ligation template has a 3′ region that is complementary to the 3′ end of the 5′ linker and a degenerate 5′ region comprising a random mix of nucleotides, such that each template may hybridize with the 5′ end of a specific mature small RNA in the population of mature small RNAs. The method further comprises ligating a 3′ linker, having a phosphate at its 5′ end, to the 3′ end of a mature small RNA (or a mature small RNA ligated to a 5′ linker) through the use of a 3′ semi-degenerate ligation template that spans the 3′ ligation junction. The 3′ semi-degenerate ligation template has a 5′ region that is complementary to the 5′ end of the 3′ linker and a degenerate 3′ region comprising a random mix of nucleotides, such that each template may hybridize with the 3′ end of a specific mature small RNA in the population of mature small RNAs. Each of the semi-degenerate ligation templates will further comprise a biotin affinity tag.

The 5′ linkers will be similar to the 5′ linkers used in the preceding examples. The 3′ linker will resemble the 5′ linker, except it will have a phosphate at its 5′ end. The 5′ and 3′ linkers may comprise ribonucleotides, deoxyribonucleotides, or a combination thereof. If the 3′ linker is a DNA-RNA chimeric, the RNA nucleotide will be at its 5′ end. If the 3′ linker forms a stem-loop structure, the stem will comprise the 3′ end of the linker and the 5′ end of the linker will be single-stranded. The 5′ linker may further comprise a RNA polymerase promoter sequence and the 3′ linker may further comprise a RNA polymerase promoter sequence in the reverse orientation.

The ligation reaction will contain a plurality of mature small RNAs, a 5′ linker, a 5′ semi-degenerate ligation template, a 3′ linker, a 3′ semi-degenerate ligation template, and T4 DNA ligase, and it will be performed under conditions as described above.

Following the ligation reaction, the ligation mixture will be transferred to a streptavidin-coated tube where the ligation product will be captured through the interaction between biotin attached to the semi-degenerate templates and streptavidin in the tube. Non-target RNAs and other reagents will be removed from the tube. After an optional wash, an elution buffer will be added to the tube and the contents of the tube will be heated to about 70° C. to release the ligation product from the templates. The eluted ligation product will be reverse transcribed into cDNA using a reverse transcriptase (such as MMLV RT) and reverse primer that is complementary to the 3, linker. The cDNA will be amplified using qPCR as described above, using a forward primer that corresponds to the 5′ linker and a reverse primer that is complementary to the 3′ linker. Alternatively, the cDNA and/or the PCR products could be amplified by transcription with a RNA polymerase into sense and antisense strands of mature small RNA/ligation product, provided that each linker has an appropriate RNA polymerase promoter sequence in the correct orientation.

### Example 13

### Genome-Wide Detection of Mature Small RNAs—Two-Step Ligation

The method described in Example 12 will be modified such that the ligation reactions are done sequentially, as diagrammed in FIG. 3. The linkers and semi-degenerate templates used in Example 12 will also be used in the example. Following a first ligation in which the 5′ linker is linked to the 5′ end of a population of mature small RNAs in the presence of the 5′ semi-degenerate ligation template, the ligation mixture will be transferred to a streptavidin-coated tube to capture the first ligation product, as described above. Then a second ligation will be performed in which the 3′ linker is ligated to the 3′ end of the first ligation product in the presence of the 3′ semi-degenerate ligation template. The second ligation mixture will be transferred to a streptavidin-coated tube to capture the ligation products, as described above. The ligation products will be reverse transcribed, amplified, and detected as described above in Example 12.

What is claimed is:

1. A method for detecting a mature small RNA, the method comprising:
a. providing a sample comprising a mature small RNA whose sequence is known;
b. ligating a 5′ linker to the 5′ end of the mature small RNA in the presence of a complementary ligation template that spans the ligation junction, whereby a ligation product is formed, the ligation product comprising the 5′ linker and the mature small RNA; and,
c. assaying the ligation product, such that the mature small RNA is detected.

2. The method of claim 1, wherein the mature small RNA is selected from the group consisting of a microRNA (miRNA), a short interfering RNA (siRNA), a repeat associated siRNA (rasiRNA), a transacting siRNA (tasiRNA), a Piwi interacting RNA (piRNA), and a 21-U RNA.

3. The method of claim 1, wherein the sample comprising a mature small RNA is selected from the group consisting of an isolated preparation of RNA, a cellular extract, an intact cell, an in vitro transcription reaction, and a chemical synthesis.

4. The method of claim 1, wherein the 5′ linker is selected from the group consisting of a DNA polynucleotide and a chimeric DNA-RNA polynucleotide having at least one ribonucleotide at the 3′ end.

5. The method of claim 4, wherein the linker further comprises a stem-loop structure, with the 5′ end forming part of the stem and the 3′ end forming a single-stranded overhang.

6. The method of claim 1, wherein the complementary ligation template comprises a 3′ region that hybridizes under stringent conditions to the 3′ end of the linker and a 5′ region that hybridizes under stringent conditions to the 5′ end of the mature small RNA.

7. The method of claim 6, wherein the ligation template is selected from the group consisting of a RNA oligonucleotide, a chimeric DNA-RNA oligonucleotide, a DNA oligonucleotide, and a DNA oligonucleotide comprising at least one PCR blocker.

8. The method of claim 7, wherein the PCR blocker is selected from the group consisting of a dideoxynucleotide, an amine group, a methyl group, a phosphate group, and carbon spacers.

9. The method of claim 1, wherein the ligation is catalyzed by a ligase, the ligase is a template-dependent ligase selected from the group consisting of T4 DNA ligase, vaccinia DNA ligase, and mammalian DNA ligases.

10. The method of claim 1, wherein the mature small RNA is a microRNA, the 5′ linker is a chimeric DNA-RNA polynucleotide of about 90 nucleotides that forms a stem loop structure, the complementary ligation template is an RNA oligonucleotide of about 14 nucleotides, and the ligase is T4 DNA ligase.

11. The method of claim 1, wherein the ligation product is assayed by a method selected from the group consisting of an amplification method and a hybridization method.

12. The method of claim 11, wherein the amplification method is selected from the group consisting of quantitative real-time PCR, quantitative end-point PCR, and standard PCR

13. The method of claim 11, wherein the hybridization method is selected from the group consisting of nucleic acid microarray, nucleic acid-coupled microsphere array, and branched DNA technology.

14. The method of claim 12, wherein the PCR method comprises a forward PCR primer that corresponds to a sequence of the 5′ linker and a reverse PCR primer that is complementary to a portion of the small RNA.

15. The method of claim 14, wherein the PCR primers comprise at least one modified nucleotide, the modified nucleotide comprises a locked nucleic acid (LNA).

16. The method of claim 1, wherein step (b) is performed in the presence of a plurality of complementary ligation templates, rather than a single ligation template, such that a plurality of mature small RNAs is detected.

17. The method of claim 16, wherein the 5′ end of each of the ligation templates is complementary to the 5′ end of a discrete mature small RNA in the plurality of mature small RNAs.

18. A method for detecting a population of mature small RNAs selected from the group consisting of known mature small RNAs and unknown mature small RNAs, the method comprising:
a. providing a sample comprising a population of mature small RNAs;
b. ligating a first linker to one end of a mature small RNA in the population of mature small RNAs in the presence of a first semi-degenerate ligation template that spans the first ligation junction, whereby a plurality of first ligation products is formed, each first ligation product comprising a first linker and a mature small RNA;
c. ligating a second linker to the small RNA end of a ligation product in the plurality of first ligation products in the presence of a second semi-degenerate ligation template that spans the second ligation junction, whereby a plurality of second ligation products is formed, each second ligation product comprising a first linker, a mature small RNA, and a second linker; and,
d. assaying the plurality of second ligation products, such that a population of mature small RNAs is detected.

19. The method of claim 18, wherein steps (b) and (c) are performed simultaneously.

20. The method of claim 18, wherein steps (b) and (c) are performed sequentially.

21. The method of claim 19, wherein the second ligation products are separated from the reaction materials before step (d).

22. The method of claim 20, wherein the first ligation products are separated from the reaction materials before step (c), and the second ligation products are separated from the reaction materials before step (d).

23. The method of claim 18, wherein the sample comprising a population of mature small RNAs is selected from the group consisting of an isolated preparation of RNA, a cellular extract, and an intact cell.

24. The method of claim 18, wherein the first linker is a 5′ linker, the first semi-degenerate ligation template is a 5′ semi-degenerate ligation template that spans the 5′ ligation junction, the second linker is a 3′ linker, and the second semi-degenerate ligation template is a 3′ semi-degenerate ligation template that spans the 3′ ligation junction.

25. The method of claim 18, wherein the first linker is a 3′ linker, the first semi-degenerate ligation template is a 3′ semi-degenerate ligation template that spans the 3′ ligation junction, the second linker is a 5′ linker, and the second semi-degenerate ligation template is a 5′ semi-degenerate ligation template that spans the 5′ ligation junction.

26. The method of claim 18, wherein the linkers and semi-degenerate ligation templates are selected from the group consisting of a RNA oligonucleotide, a DNA oligonucleotide, and a chimeric RNA-DNA oligonucleotide.

27. The method of claim 18, wherein the 5′ semi-degenerate ligation template comprises a 3′ region that hybridizes under stringent conditions to the 3′ end of the 5′ linker, and a degenerate 5′ region comprising a random mix of nucleotides, such that each ligation template hybridizes with the 5′ end of a discrete small RNA in the population of small RNAs.

28. The method of claim 18, wherein the 3′ semi-degenerate ligation template comprises a 5′ region that hybridizes under stringent conditions to the 5′ end of the 3′ linker, and a degenerate 3′ region comprising a random mix of nucleotides, such that each ligation template hybridizes with the 3′ end of a discrete small RNA in the population of small RNAs.

29. The method of claim 18, wherein the 5′ and 3′ semi-degenerate ligation templates further comprise a separation tag selected from the group consisting of biotin, digoxigenin, a fluorophore, and a magnetic particle.

30. The method of claim 18, wherein the 31 linker comprises a 5′ phosphate group.

31. The method of claim 18, wherein the 5′ linker further comprises a stem-loop structure, with the 5′ end forming part of the stem and the 3′ end forming a single-stranded overhang, and the 3′ linker further comprises a stem-loop structure, with the 3′ end forming part of the stem and the 5′ end forming a single-stranded overhang.

32. The method of claim 18, wherein the ligation reactions are catalyzed by a ligase, the ligase is a template-dependent ligase selected from the group consisting of T4 DNA ligase, vaccinia DNA ligase, and mammalian DNA ligases.

33. The method of claim 18, wherein the plurality of second ligation products is assayed by converting the ligation products into DNA copies and amplifying the DNA copies.

34. The method of claim 33, wherein the DNA copies are generated using a method comprising a reverse transcriptase and a reverse primer that is complementary to a region of the 3′ linker.

35. The method of claim 33, wherein the DNA copies are amplified using a PCR method comprising a forward PCR primer that corresponds to a sequence of the 5′ linker and a reverse PCR primer that is complementary to a sequence of the 3′ linker.

36. The method of claim 33, wherein the DNA copies are amplified using a transcription method comprising at least one RNA polymerase, provided that the 5′ linker and the 3′ linker each further comprise a RNA polymerase promoter sequence in the proper orientation.

37. A kit for detecting a mature small RNA whose sequence is known, the kit comprising a 5′ linker for ligating to the 5′ end of the mature small RNA, a ligation template that is complementary to the junction between the 5′ linker and the mature small RNA, a ligase, and instructions for using the kit.

38. The kit of claim 37, wherein the 5′ linker is selected from the group consisting of a DNA polynucleotide and a chimeric DNA-RNA polynucleotide having at least one ribonucleotide at the 3′ end.

39. The kit of claim 38, wherein the 5′ linker further comprises a stem-loop structure, with the 5′ end forming part of the stem and the 3′ end forming a single-stranded overhang.

40. The kit of claim 37, wherein the complementary ligation template comprises a 3′ region that hybridizes under stringent conditions to the 3′ end of the 5′ linker and a 5′ region that hybridizes under stringent conditions to the 5′ end of the mature small RNA

41. The kit of claim 37, wherein the complementary ligation template is selected from the group consisting of a RNA oligonucleotide, a chimeric DNA-RNA oligonucleotide, a DNA oligonucleotide, and a DNA oligonucleotide comprising at least one PCR blocker, wherein the PCR blocker is selected from the group consisting of a dideoxynucleotide, an amine group, a methyl group, a phosphate group, and carbon spacers.

42. The kit of claim 37, wherein the ligase is a template-dependent ligase selected from the group consisting of T4 DNA ligase, vaccinia DNA ligase, and mammalian DNA ligases.

43. The kit of claim 37 further comprising a forward PCR primer that corresponds to a sequence of the 5′ linker, a reverse PCR primer that is complementary to a portion of the small RNA, and a set of reagents for quantitative PCR.

44. The kit of claim 37, further comprising a plurality of complementary ligation templates for the detection of a plurality of mature small RNAs whose sequences are known, wherein the 5′ end of each of the ligation templates is complementary to the 5′ end of a discrete small RNA in the plurality of mature small RNAs.

45. A kit for detecting a population of mature small RNAs selected from the group consisting of known small RNAs and unknown small RNAs, the kit comprising a 5′ linker for ligating to the 5′ end of a mature small RNA, a 3′ linker for ligating to the 3′ end of a mature small RNA, a 5′ semi-degenerate ligation template that is complementary to the 5′ ligation junction, a 3′ semi-degenerate complementary ligation template that is complementary to the 3′ ligation junction, a ligase, and instructions for using the kit.

46. The kit of claim 45, wherein the 5′ semi-degenerate ligation template comprises a 3′ region that hybridizes under stringent conditions to the 3′ end of the 5′ linker, and a degenerate 5′ region comprising a random mix of nucleotides, such that each semi-degenerate ligation template hybridizes with the 5′ end of a discrete mature small RNA in the population of mature small RNAs.

47. The kit of claim 45, wherein the 3′ semi-degenerate ligation template comprises a 5′ region that hybridizes under stringent conditions to the 5′ end of the 3′ linker, and a degenerate 3′ end sequence comprising a random mix of nucleotides, such that each semi-degenerate ligation template hybridizes with the 3′ end of a discrete mature small RNA in the population of mature small RNAs.

48. The kit of claim 45, wherein the linkers and semi-degenerate ligation templates are selected from the group consisting of a RNA oligonucleotide, a DNA oligonucleotide, and a chimeric RNA-DNA oligonucleotide.

49. The kit of claim 45, wherein the 5′ and 3′ semi-degenerate ligation templates further comprise a separation tag selected from the group consisting of biotin, digoxigenin, a fluorophore, and a magnetic particle.

50. The kit of claim 45, wherein the 3′ linker comprises a 5′ phosphate group.

51. The kit of claim 45, wherein the 5′ linker further comprises a stem-loop structure, with the 5′ end forming part of the stem and the 3′ end forming a single-stranded overhang, and the 3′ linker further comprises a stem-loop structure, with the 3′ end forming part of the stem and the 5′ end forming a single-stranded overhang.

52. The kit of claim 45, wherein the ligase is a template-dependent ligase selected from the group consisting of T4 DNA ligase, vaccinia DNA ligase, and mammalian DNA ligases.

53. The kit of claim 45 further comprising a reverse transcriptase and a reverse primer that is complementary to a sequence of the 3′ linker.

54. The kit of claim 45 further comprising a forward PCR primer that corresponds to a sequence of the 5′ linker, a reverse PCR primer that is complementary to a portion of the 3′ linker, and a set of reagents for quantitative PCR.


SEQUENCE LISTING (59 sequences)

SEQ ID NO: 1 - RNA, 22 nt, Homo sapiens
  misc_feature (1)..(1): PHOSPHATE
ugagguaguagguuguauaguu

SEQ ID NO: 2 - RNA, 22 nt, Homo sapiens
ugagguaguagguuguauaguu

SEQ ID NO: 3 - DNA, 93 bp, Artificial
  5′ Linker
ggcaggtaatacgactcactataggtcgagagtcagggagcactccagctgcgaccaggagaggtgtctcagcagagtacctgccagcaactg

SEQ ID NO: 4 - RNA, 18 nt, Artificial
  Ligation template
uacuaccucacaguugcu

SEQ ID NO: 5 - RNA, 18 nt, Artificial
  Ligation template
cgugcugcuacaguugcu

SEQ ID NO: 6 - DNA, 23 bp, Artificial
  Reverse primer
aactatacaacctactacctcac

SEQ ID NO: 7 - DNA, 21 bp, Artificial
  Forward primer
ggcaggtaatacgactcacta

SEQ ID NO: 8 - RNA, 22 nt, Mus musculus
uagcagcacguaaauauuggcg

SEQ ID NO: 9 - DNA, 23 bp, Artificial
  Reverse primer
cgccaatatttacgtgctgctac

SEQ ID NO: 10 - DNA, 22 bp, Artificial
  Reverse primer
cgccaatatttacgtgctgcta

SEQ ID NO: 11 - DNA, 22 bp, Artificial
  Reverse primer
cgccaatatttacgtgctgcta

SEQ ID NO: 12 - DNA, 93 bp, Artificial; identical to SEQ ID NO: 3
  5′ Linker

SEQ ID NO: 13 - DNA, 91 bp, Artificial
  5′ Linker
ggcaggtaatacgactcactataggtcgagagtcagggagcactccagctgcgaccaggagaggtgtctcagcagagtacctgcccaatga

SEQ ID NO: 14 - RNA, 14 nt, Artificial
  Ligation template
ugcugcuaucauug

SEQ ID NO: 15 - RNA, 16 nt, Artificial
  5′ Linker
cgugcugcuaucauug

SEQ ID NO: 16 - DNA, 93 bp, Artificial
  5′ Linker
ggcaggtaatacgactcactataggtcgagagtcagggagcactccagctgcgaccaggagaggtgtctcagcagagtacctgccagcaatga

SEQ ID NO: 17 - RNA, 16 nt, Artificial
  Ligation template
ugcugcuaucauugcu

SEQ ID NO: 18 - RNA, 18 nt, Artificial
  Ligation template
cgugcugcuaucauugcu

SEQ ID NO: 19 - DNA, 95 bp, Artificial
  5′ Linker
ggcaggtaatacgactcactataggtcgagagtcagggagcactccagctgcgaccaggagaggtgtctcagcagagtacctgccagagcaatga

SEQ ID NO: 20 - RNA, 20 nt, Artificial
  Ligation template
cgugcugcuaucauugcucu

SEQ ID NO: 21 - DNA, 97 bp, Artificial
  5′ Linker
ggcaggtaatacgactcactataggtcgagagtcagggagcactccagctgcgaccaggagaggtgtctcagcagagtacctgcctcagagcaatga

SEQ ID NO: 22 - RNA, 24 nt, Artificial
  Ligation template
uacgugcugcuaucauugcucuga

SEQ ID NO: 23 - DNA, 91 bp, Artificial
  5′ Linker
ggcaggtaatacgactcactataggtcgagagtcagggagcactccagctgcgaccaggagaggtgtctcagcagagtacctgcccaacag

SEQ ID NO: 24 - RNA, 14 nt, Artificial
  Ligation template
ugcugcuacuguug

SEQ ID NO: 25 - RNA, 22 nt, Homo sapiens
uagcuuaucagacugauguuga

SEQ ID NO: 26 - RNA, 14 nt, Artificial
  Ligation template
auaagcuacuguug

SEQ ID NO: 27 - DNA, 23 bp, Artificial
  Reverse primer
tcaacatcagtctgataagctac

SEQ ID NO: 28 - RNA, 21 nt, Homo sapiens
uggaauguaaagaaguaugua

SEQ ID NO: 29 - RNA, 14 nt, Artificial
  Ligation template
acauuccacuguug

SEQ ID NO: 30 - DNA, 22 bp, Artificial
  Reverse primer
tacatacttctttacattccac

SEQ ID NO: 31 - RNA, 23 nt, Homo sapiens
gcaaagcacacggccugcagaga

SEQ ID NO: 32 - RNA, 14 nt, Artificial
  Ligation template
ugcuuugccuguug

SEQ ID NO: 33 - DNA, 24 bp, Artificial
  Reverse primer
tctctgcaggccgtgtgctttgcc

SEQ ID NO: 34 - RNA, 21 nt, Homo sapiens
aucacauugccagggauuacc

SEQ ID NO: 35 - RNA, 15 nt, Artificial
  Ligation template
caaugugaucuguug

SEQ ID NO: 36 - DNA, 22 bp, Artificial
  Reverse primer
ggtaatccctggcaatgtgatc

SEQ ID NO: 37 - RNA, 22 nt, Homo sapiens
cagugcaauguuaaaagggcau

SEQ ID NO: 38 - RNA, 14 nt, Artificial
  Ligation template
uugcacugcuguug

SEQ ID NO: 39 - DNA, 23 bp, Artificial
  Reverse primer
atgcccttttaacattgcactgc

SEQ ID NO: 40 - RNA, 14 nt, Artificial
  Ligation template
cuaccucacuguug

SEQ ID NO: 41 - RNA, 20 nt, Arabidopsis thaliana
ugacagaagagagugagcac

SEQ ID NO: 42 - RNA, 14 nt, Artificial
  Ligation template
uucugucacuguug

SEQ ID NO: 43 - DNA, 21 bp, Artificial
  Reverse primer
gtgctcactctcttctgtcac

SEQ ID NO: 44 - RNA, 21 nt, Arabidopsis thaliana
agaaucuugaugaugcugcau

SEQ ID NO: 45 - RNA, 15 nt, Artificial
  Ligation template
caagauucucuguug

SEQ ID NO: 46 - DNA, 22 bp, Artificial
  Reverse primer
atgcagcatcatcaagattctc

SEQ ID NO: 47 - RNA, 21 nt, Arabidopsis thaliana
cagccaaggaugacuugccga

SEQ ID NO: 48 - RNA, 14 nt, Artificial
  Ligation template
cuuggcugcuguug

SEQ ID NO: 49 - DNA, 22 bp, Artificial
  Reverse primer
tcggcaagtcatccttggctgc

SEQ ID NO: 50 - RNA, 21 nt, Arabidopsis thaliana
uuuggauugaagggagcucua

SEQ ID NO: 51 - RNA, 15 nt, Artificial
  Ligation template
caauccaaacuguug

SEQ ID NO: 52 - DNA, 22 bp, Artificial
  Reverse primer
tagagctcccttcaatccaaac

SEQ ID NO: 53 - RNA, 21 nt, Arabidopsis thaliana
ugauugagccgugucaauauc

SEQ ID NO: 54 - RNA, 14 nt, Artificial
  Ligation template
cucaaucacuguug

SEQ ID NO: 55 - DNA, 22 bp, Artificial
  Reverse primer
gatattgacacggctcaatcac

SEQ ID NO: 56 - RNA, 21 nt, Arabidopsis thaliana
ucggaccaggcuucauucccc

SEQ ID NO: 57 - RNA, 14 nt, Artificial
  Ligation template
ugguccgacuguug

SEQ ID NO: 58 - DNA, 22 bp, Artificial
  Reverse primer
ggggaatgaagcctggtccgac

SEQ ID NO: 59 - RNA, 14 nt, Artificial
  Ligation template
nnnnnnnacuguug